(19) United States
(12) Patent Application Publication
(10) Pub. No.: US 2023/0073245 A1
(43) Pub. Date: Mar. 9, 2023

(54) TOLL-LIKE RECEPTOR AGONIST FORMULATIONS AND METHODS OF USE

(71) Applicants: The University of Chicago, Chicago, IL (US); Central Michigan University, Mount Pleasant, MI (US)

(72) Inventors: Aaron ESSER-KAHN, Chicago, IL (US); Saikat MANNA, Chicago, IL (US); WenJun DU, Mount Pleasant, MI (US); Sampa MAITI, Mount Pleasant, MI (US)

(73) Assignees: The University of Chicago, Chicago, IL (US); Central Michigan University, Mount Pleasant, MI (US)

(21) Appl. No.: 17/758,264

(22) PCT Filed: Jan. 13, 2021
(86) PCT No.: PCT/US2021/070024

§ 371 Date: Jun. 30, 2022

Related U.S. Application Data
(60) Provisional application No. 62/960,520, filed on Jan. 13, 2020.

(51) Int. Cl. A61K 9/51; A61K 47/69

(52) U.S. Cl. CPC A61K 9/5146; A61K 9/5161; A61K 47/6929; A61K 9/5169

(57) ABSTRACT

Aspects of the disclosure relate to nanoparticle formulations and methods for generating nanoparticles. Embodiments include nanoparticles comprising an amphiphile and a polymer co-assembly agent. In some cases, polymers for use in therapeutic delivery are described. In some embodiments, the disclosed methods and compositions involve TLR agonists and formulations thereof capable of activating an immune response. Certain aspects relate to nanoparticles comprising linked TLR agonists for use in immunotherapy.

### CROSS-REFERENCE TO RELATED APPLICATIONS

This application claims the benefit of priority of U.S. Provisional Patent Application No. 62/960,520 filed Jan. 13, 2020, which is hereby incorporated by reference in its entirety.

### BACKGROUND

This invention was made with government support under 3U01A1124286-0551 awarded by the National Institutes of Health. The government has certain rights in the invention.

### Field of the Invention

This disclosure relates generally to the fields of molecular biology, chemistry, and immunology.

### Background

Immune checkpoint blockade has provided evidence that the immune system can be targeted for cancer therapy.¹⁻³ A large fraction of patients do not respond to existing immune checkpoint blockade therapies.^{1,4,5} Thus, other immune-modulatory pathways are being investigated to target unresponsive immunologically “cold” tumors where the immuno-suppressive environment prevents initial priming of anti-tumor T-cells.⁶ One example is delivery of immune signals such as pathogen associated molecular patterns (PAMPs) to activate pattern recognition receptors (PRRs) in dendritic cells (DCs) and macrophages, which may serve to initiate downstream immune response to attract activated T-cells.^{7,8}

Toll-Like Receptors (TLRs) are important and well-studied PRRs.⁹ Targeting of TLRs using one or more agonists has been used to enhance T-cell response.¹⁰ However, hematological toxicity of TLR agonists due to systemic cytokine production is a key barrier to effective implementation of TLR-mediated immunotherapy and often hinders clinical translation due to unacceptable levels of toxicity.¹¹ This systemic toxicity is attributed to diffusion from the site of injection into the bloodstream.¹¹⁻¹⁴ This effect is compounded when multiple agonists are admixed in formulations since each agonist has a different PK/PD profile and different diffusion rates.¹⁵

Recognized herein is a need for methods and compositions for effective delivery of one or more agents (including, e.g., TLR agonists) with limited toxicity.

### SUMMARY OF THE DISCLOSURE

Disclosed herein are methods and compositions related to formulation and delivery of agents (e.g., therapeutic agents, diagnostic agents, etc.). Certain embodiments of the present disclosure relate to novel nano-medicines designed to efficiently deliver linked immunotherapeutic agents for treatment of solid tumors. In some embodiments, nanoparticles of the present disclosure comprise amphiphiles formulated in a stable, self-assembling nanostructure. Nanoparticles disclosed herein may be used to provide an agent to an individual, thereby improving targeted delivery of the agent and reducing systemic toxicity. In some embodiments, TLR agonists (e.g., linked TLR agonists) are provided using nanoparticles and delivery systems of the present disclosure.

Embodiments of the disclosure include nanoparticles, polymers, oligomers, pharmaceutical compositions, pharmaceutical formulations, methods for making a nanoparticle, methods for making a polymer, methods for formulating a nanoparticle, methods for delivering an agent to an individual, methods for delivering a nanoparticle to an individual, methods of providing a therapy, methods of synthesizing a polymer, methods of functionalizing a polymer, methods of treating a condition, methods for treating cancer, methods for treating an autoimmune condition, methods for treating a viral infection, and methods for treating an allergic disorder. In some embodiments, any one or more of these embodiments or elements may be excluded.

Compositions (e.g., nanoparticles) of the present disclosure can include one or more of the following: an amphiphile, an amphiphilic group, a hydrophilic linker, a hydrophobic group, a co-assembly agent, a functionalized polymer, a hydrophilic polymer, a co-assembly agent linker, a polypeptide, a pattern recognition receptor (PRR) agonist, a NOD-like receptor agonist, a RIG-I-like receptor agonist, a STING agonist, a TLR agonist, a TLR2/6 agonist, Pam₂CSK₄, Pam₃CSK₄, MALP-2, FSL-1, a TLR 7 agonist, a TLR8 agonist, 2Bxy, a maleimide moiety, a polyethylene glycol (PEG) moiety, a triazole moiety, a functionalized polysaccharide, a functionalized poly(orthoester), an olefinic group, an oleyl group, a therapeutic agent, or a diagnostic agent. It is contemplated that any one of these components or elements may be excluded from embodiments of the disclosed compositions.

Methods can include at least 1, 2, 3, 4, 5, 6, 7, 8, 9, or more of the following steps: providing an agent, providing a therapy, treating a condition, preventing a condition, administering a composition, diagnosing a condition, generating a nanoparticle, generating an amphiphile, synthesizing a polymer, and functionalizing a polymer. It is contemplated that any one of these steps may be excluded from embodiments of the disclosed methods.

Disclosed herein, in certain aspects, is a nanoparticle comprising an amphiphile and a nanoparticle co-assembly agent. In some embodiments, the amphiphile is of formula (I): A-B-C, wherein A is an amphiphilic group, B is a hydrophilic linker, and C is a first hydrophobic group. In some embodiments, the nanoparticle co-assembly agent is of formula (II): X-(Y-Z)ₘ, wherein X is a functionalized hydrophilic polymer, Y is a co-assembly agent linker, Z is a second hydrophobic group, and m is an integer ranging from 1 to 500 (or any range or value derivable therein). In some embodiments, m is 1, 2, 3, 4, 5, 6, 7, 8, 20, 30, 40, 50, 60, 70, 80, 90, 100, 200, 300, 400, 500, or any range or value derivable therein. In some embodiments, the nanoparticle is a self-assembled nanoparticle. In some embodiments, the amphiphile and the nanoparticle co-assembly agent are not covalently attached to each other. In some embodiments, A comprises a polypeptide. The polypeptide may be a hydrophilic polypeptide. In some embodiments, A comprises an amphiphilic pattern recognition receptor (PRR) agonist. The amphiphilic PRR agonist may be a NOD-like receptor agonist, a RIG-I-like receptor agonist, a STING agonist, or a TLR agonist. In some embodiments, the amphiphilic PRR agonist is an amphiphilic TLR agonist, which may be a TLR2/6 agonist. In some embodiments, the amphiphilic TLR agonist is Pam₂CSK₄, Pam₃CSK₄, MALP-2, or FSL-1. In some embodiments, the amphiphilic TLR agonist is Pam₂CSK₄.

In some embodiments, C comprises a hydrophobic PRR agonist. The hydrophobic PRR agonist may be a NOD-like receptor agonist, a RIG-I-like receptor agonist, a STING agonist, or a TLR agonist. In some embodiments, the hydrophobic PRR agonist is a hydrophobic TLR agonist, which may be a TLR7 agonist or a TLR8 agonist. In some embodiments, the hydrophobic PRR agonist is 2Bxy. In some embodiments, B comprises at least one of a maleimide moiety, a polyethylene glycol (PEG) moiety, and a triazole moiety. In some embodiments, B comprises a PEG moiety, wherein the PEG moiety has between 3 and 7 ethyleneoxy units. In some embodiments, the PEG moiety has 3, 4, 5, 6, or 7 ethyleneoxy units. The PEG moiety may have 5 ethyleneoxy units.

In some embodiments, X is a functionalized PEG or a functionalized polysaccharide. In some embodiments, X is a pegylated polysaccharide or a monosaccharide poly(orthoester). In some embodiments, Y comprises at least one of a maleimide moiety, a PEG moiety, and a triazole moiety. In some embodiments, Y comprises a triazole moiety. In some embodiments, Z is an alkyl or olefinic group having at least 10 carbon atoms, which may be an oleyl group. In some embodiments, the amphiphile is of formula (III):

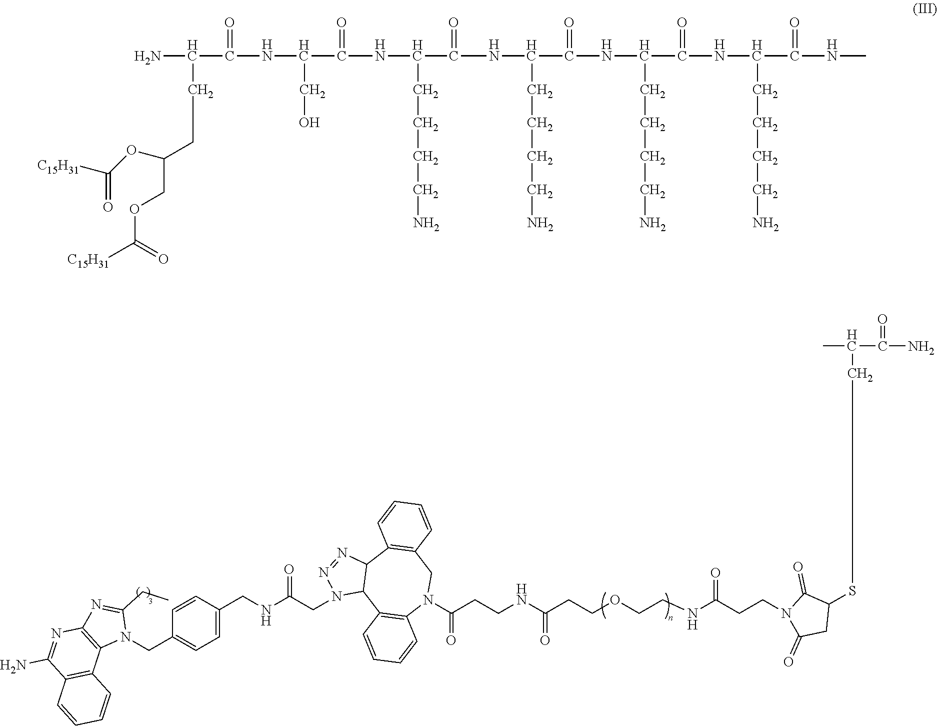

wherein n is an integer ranging from 3 to 7. In some embodiments, n is 3, 4, 5, 6, or 7.

In some embodiments, the amphiphile is of formula (IV):

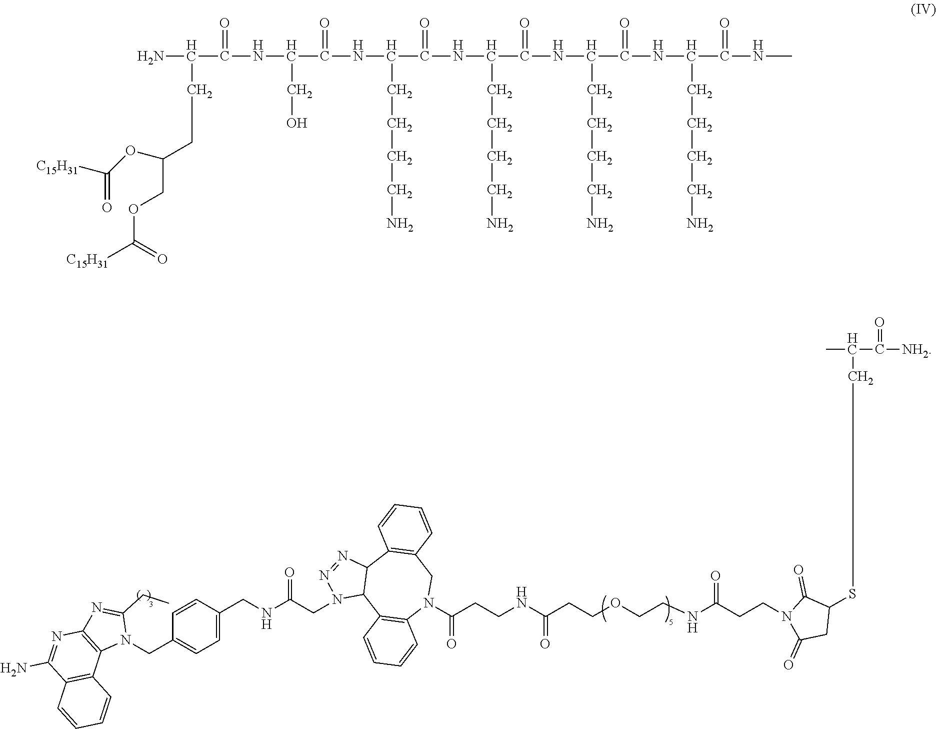

In some embodiments, the nanoparticle co-assembly agent is of formula (V):

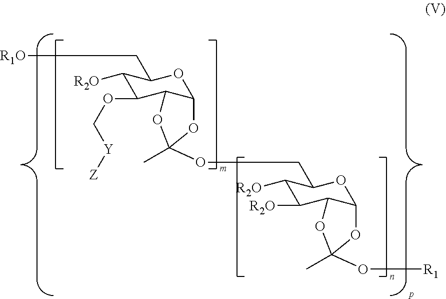

wherein R₁ is alkyl, acyl, or H, R2 is alkyl, acyl, or H, m is an integer ranging from 1 to 10, n is an integer ranging from 0 to 10, and p is an integer ranging from 2 to 500. The ratio of m:n may range from 1:0 to 1:10, which in some embodiments may be 1:5. In some embodiments, Y includes at least one of a maleimide moiety, a PEG moiety, and a triazole moiety. In some aspects, the PEG moiety comprises from 2 to 20 ethyleneoxy units. In some embodiments, Z is an alkyl or acyl group having at least 10 carbon atoms. In some aspects, Z includes at least one degree of unsaturation.

In some embodiments, the nanoparticle co-assembly agent is of formula (VI):

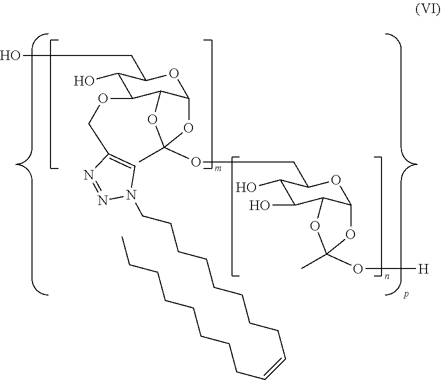

Aspects of the disclosure relate to a method of making a nanoparticle of the present disclosure comprising providing the amphiphile and the nanoparticle co-assembly agent in a salt solution. In some embodiments, the salt solution is a balanced salt solution. In some embodiments, the salt solution is a buffered salt solution. The salt solution may be phosphate buffered saline (PBS). In some embodiments, the method for making the nanoparticle comprises providing the amphiphile and the nanoparticle co-assembly agent in the salt solution for at least 24 hours. In some embodiments, the method further comprises subjecting the salt solution to dialysis.

Disclosed herein, in some aspects, is a method of delivering an agent to an individual, the method comprising providing a nanoparticle described herein to the individual, wherein the amphiphile comprises the agent. In some embodiments, the nanoparticle is provided intravenously. The agent may be an imaging agent or a therapeutic agent. An imaging agent may be a fluorescent agent, a chemiluminescent agent, or a radiocontrast agent. A therapeutic agent may be an anti-viral agent, a chemotherapeutic, an immunotherapeutic, or an immunostimulatory agent. The method may comprise treating a condition in an individual, which may be, for example, a viral infection, a neoplasm, an allergic disorder, or an autoimmune condition. In some embodiments, treating a condition in an individual further comprises providing a therapy. In some embodiments, the nanoparticle and the therapy are provided substantially simultaneously. In some embodiments, the nanoparticle and the therapy are provided sequentially. The therapy may be, for example, an anti-viral therapy, a chemotherapy, an immunotherapy, a radiotherapy, or a vaccine.

Certain aspects of the present disclosure relate to polymers and polymer formulations. In some embodiments, disclosed herein is a polymer of formula (VII):

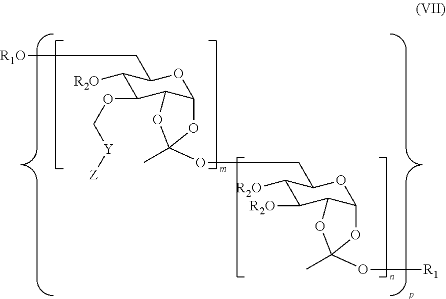

wherein R₁ is alkyl, acyl, or H, R₂ is alkyl, acyl, or H, m is an integer ranging from 1 to 10, n is an integer ranging from 0 to 10, Y is a linker, Z is a hydrophobic group, and p is an integer ranging from 2 to 500.

Y may include at least one of a maleimide moiety, a PEG moiety, and a triazole moiety. In some aspects, the PEG moiety comprises from 2 to 20 ethyleneoxy units. The PEG moiety may comprise 2, 3, 4, 5, 6, 7, 8, 9, 10, 11, 12, 13, 14, 15, 16, 17, 18, 19, or 20 ethyleneoxy units. In some embodiments, Y comprises a triazole moiety. In some embodiments, Z is an alkyl or acyl group having at least 10 carbon atoms. In some aspects, Z includes at least one degree of unsaturation. In some embodiments, Z is an oleyl group. In some embodiments, the ratio of m:n ranges from 1:0 to 1:10, which may be 1:5.

In some embodiments, the polymer is of formula (VIII):

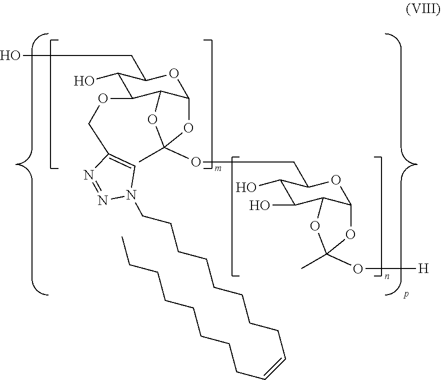

Embodiments of the present disclosure are directed to pharmaceutical compositions comprising a nanoparticle of the present disclosure and/or a polymer of the present disclosure together with a pharmaceutically acceptable excipient.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Example section are understood to be embodiments that are applicable to all aspects of the technology described herein.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows an ¹H-NMR spectrum of toll like receptor agonist (7/8a) azide 2Bxy.

FIG. 2 shows an ¹H-NMR spectrum of oleyl bromide.

FIG. 3 shows a ¹³C-NMR spectrum of oleyl bromide.

FIG. 4 shows an ¹H-NMR spectrum of oleyl azide.

FIG. 5 shows a ¹³C-NMR spectrum of oleyl azide.

FIG. 6 shows an ¹H-NMR spectrum of monomer I as described in Example 1.

FIG. 7 shows a ¹³C-NMR spectrum of monomer I as described in Example 1.

FIG. 8 shows an ¹H-NMR spectrum of nonfunctional monomer II as describd in Example 1.

FIG. 9 shows a ¹³C-NMR spectrum of nonfunctional monomer II as describd in Example 1.

FIGS. 10A-C show ¹H-NMR spectra of SPOE (FIG. 10A), oleyl grafted SPOE (OL-SPOE) (FIG. 10B), and deprotected oleyl grafted SPOE (OL-DSPOE) (FIG. 10C).

FIG. 11 shows a GPC analysis of SPOE, OL-SPOE, and OL-DSPOE.

FIGS. 12A-12I show results from nanoparticle generation described in Example 2. FIGS. 12A-12I show 2/6_7a self-assembly in water for 3 days (FIG. 12A) and 8 weeks (FIG. 12B), 2/6_7a self-assembly in PBS for 3 days (FIG. 12C) and 8 weeks (FIG. 12D), 2/6_7a self-assembly in the presence of sugar amphiphile for 3 days (FIG. 12E) and 8 weeks (FIG. 12F), high resolution of images of 2/6_7a self-assembly after 8 weeks in water (FIG. 12G) and PBS (FIG. 12H), and high resolution images of representative SPA particles (FIG. 121).

FIGS. 13A-F show results from in vitro studies described in Example 3. FIG. 13A shows NF-κB activity in RAW Blue macrophages. FIGS. 13B-13F show secretion, on incubation of agonists with BMDCs at 100 nM concentration, with TNF-α (FIG. 13B), CCl2 (FIG. 13C), IL-6 (FIG. 13D), IL-12p70 (FIG. 13E), and IL-10 (FIG. 13F). DSPOE: oleyl-conjugated deprotected sugar poly(orthoester), 2/6a +7a: unlinked mixture, 2/6a +7a_AZ: unlinked mixture containing 2Bxy-azide, 2/6_7a: Linked heterodimer, SPA: self-formulating PRR agonist. t-test, *p<0.05, **p<0.01, ***p<0.001, ****p<0.0001, statistical significance calculated against respective SPA samples.

FIG. 14 shows variation of in vitro SPA activity over time.

FIGS. 15A-I show results from in vivo hematological toxicity analyses described in Example 4. FIG. 15A shows a schematic of the in vivo dosing and analysis regimen. FIGS. 15B-I show results from hematological analysis of white blood cell count (FIG. 15B), lymphocyte count (FIG. 15C), monocyte count (FIG. 15D), neutrophil count (FIG. 15E), thrombocyte count (FIG. 15F), red blood cell count (FIG. 15G), hemoglobin levels (FIG. 15H), and neutrophil-to-lymphocyte ratio (FIG. 151).

FIGS. 16A-J show additional results from in vivo toxicity analyses described in Example 4. FIGS. 16A-H show results from hematological analysis of white blood cell count (FIG. 16A), lymphocyte count (FIG. 16B), monocyte count (FIG. 16C), neutrophil count (FIG. 16D), red blood cell count (FIG. 16E), hemoglobin levels (FIG. 16F), thrombocyte count (FIG. 16G), and neutrophil-to-lymphocyte ratio (FIG. 16H). FIGS. 16I shows representative spleens from each treatment group. FIG. 16J shows results from spleen area analysis.

FIGS. 17A-L show results from in vivo SPA efficacy analysis described in Example 4. FIG. 17A shows a Kaplan-Meier survival analysis of mouse treated with PBS, unlinked, linked or SPA formulations. FIG. 17B shows growth curves for tumor growth. FIG. 17C shows representative PBS treated and SPA treated mice on day 17 post tumor inoculation. FIG. 17D shows representative tumors extracted from mice on day 17. FIGS. 17E-I show results from flow cytometry analysis of CD45+ cells (FIG. 17E), percentage of CD8+ cells per total CD45+ cells (FIG. 17F), percentage of natural killer cells per total CD45+ cells (FIG. 17G), ratio of M1 to M2 macrophages. (FIG. 17H), percentage of MDSC cells per total CD45+ cells (FIG. 17I). FIGS. 17J-L show results from flow cytometry analysis from ex vivo stimulated splenocytes of percentage of IFN-g secreting CD8+ splenocytes (FIG. 17J), percentage of TNF-a secreting CD8+ splenocytes (FIG. 17K), and percentage of dual IFN-g and TNF-a secreting CD8+ splenocytes (FIG. 17L).

FIGS. 18A and 18B show results from nanoparticle synthesis experiments described in Example 5.

FIGS. 19A-19C show results from the systemic cytokine analysis describes in Example 6. A time-course analysis of systemic cytokine secretion is shown for TNF-α (FIG. 19A) and IL-6 (FIG. 19B). FIG. 19C shows change of weight in treated animals 24 hours post-agonist injection (day 9). Statistical analyses were performed using ANOVA with Tukey's multiple comparisons test.

### DESCRIPTION OF ILLUSTRATIVE EMBODIMENTS

Linked TLR agonists have been reported to have immunostimulatory effects in vaccines; however, their application in immunotherapy is yet to be explored.^{10,16,17} Nano-encapsulation of agents (e.g., within or around a nanoparticle) is one strategy for in vivo delivery. However, precise ratio-metric co-encapsulation of multiple molecules with varied polarities remains a significant challenge. It was hypothesized that, by rational linker design with proper hydrophile/lipophile balance, amphiphilic agonist heterodimers could be generated to mediate supra-molecular assemblies.¹⁹⁻²⁶ In one example, the inventors discovered that, when admixed with an amphiphilic TLR agonist heterodimer, deprotected glucose polymers functionalized with oleyl groups act as a nano-structuring agent resulting in controlled nano-formulations. In contrast to many other formulation methods, this nano-formulation uses the agonist itself for self-assembly and a small amount of molecular surfactant leading to very high loading of agonist in each particle. This “self-formulating” assembly leads to enhanced therapeutic efficacy and altered bioavailability to reduce toxicity.

The meaning of certain terms as intended is defined herein below.

### I. DEFINITIONS

Throughout this application, the term “about” is used to indicate that a value includes the inherent variation of error for the measurement or quantitation method.

As used herein, the term “agonist” refers to a molecule that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another molecule so that the other molecule directly binds to the receptor. An agonist may be referred to as an agonist of a particular receptor or family of receptors (e.g., a TLR agonist or a TNF/R agonist).

As used herein, the term “self-assembled” is used to describe a composition or formulation, such as, for example, a nanoparticle, a micelle, or other nano-structure, wherein the structure was formed from multiple components of a system spontaneously acquiring non-random special arrangements to form a larger, functional unit. For example, a self-assembled nanoparticle comprising an amphiphile and a co-assembly agent may describe a nanoparticle which was generated from the amphiphile and the co-assembly agent spontaneously forming the nanoparticle under appropriate conditions (e.g., appropriate buffer conditions, appropriate salt conditions, etc.).

As used herein, the term “nanoparticle” refers to a particle having at least one dimension in the range of about 1 nM to about 1000 nM, including any integer or non-integer value between 1 nm and 1000 nm (including about 1, 2, 5, 10, 20, 50, 60, 70, 80, 90, 100, 200, 500, and 1000 nm, and any range or value derivable therein). In some embodiments, the nanoparticle is an organic nanoparticle.

“Individual,” “subject,” and “patient” are used interchangeably and can refer to a human or non-human.

The terms “lower,” “reduced,” “reduction,” “decrease,” or “inhibit” are all used 30 herein generally to mean a decrease by a statistically significant amount. However, for avoidance of doubt, “lower,” “reduced,” “reduction,” “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “alkyl,” by itself or as part of another substituent, means, unless otherwise stated, a linear (i.e. unbranched) or branched carbon chain, which may be fully saturated, mono- or polyunsaturated. An unsaturated alkyl group is one having one or more double bonds or triple bonds. Saturated alkyl groups include those having one or more carbon-carbon double bonds (alkenyl, also olefinic) and those having one or more carbon-carbon triple bonds (alkynyl). The groups, —CH₃ (Me), —CH₂CH₃ (Et), —CH₂CH₂CH₃ (n-Pr), —CH(CH₃)₂ (iso-Pr), —CH₂CH₂CH₂CH₃ (n-Bu), —CH(CH₃)CH₂CH₃ (sec-butyl), —CH₂CH(CH₃(₂ (iso-butyl), —C(CH₃)₃ (tert-butyl), —CH₂C(CH₃)₃ (neo-pentyl), are all non-limiting examples of alkyl groups.

The term “oleyl” means a monounsaturated hydrocarbon substituent having eighteen carbons.

The term “aryl” means a polyunsaturated, aromatic, hydrocarbon substituent. Aryl groups can be monocyclic or polycyclic (e.g., 2 to 3 rings that are fused together or linked covalently). The term “heteroaryl” refers to an aryl group that contains one to four heteroatoms selected from N, O, and S. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkyl sulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkyl sulfonyl, aryl sulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “monosaccharide” refers to a cyclized monomer unit based on a compound having a chemical structure H(CHOH)ₙC(═O)(CHOH)ₘH wherein n+m is 4 or 5. Thus, monosaccharides include, but are not limited to, aldohexoses, aldopentoses, ketohexoses, and ketopentoses such as arabinose, lyxose, ribose, xylose, ribulose, xylulose, allose, altrose, galactose, glucose, gulose, idose, mannose, talose, fructose, psicose, sorbose, and tagatose. The term “polysaccharide” refers to a polymer comprising two or more individual monosaccharide units.

Suitable pharmaceutically acceptable salts may also be formed by reacting the agents of the invention with an organic base such as methylamine, ethylamine, ethanolamine, lysine, ornithine and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

The phrase “degree of unsaturation” refers to the total number of double bond equivalents and ring structures in a moiety. For example, a moiety with one degree of unsaturation includes one double bond or one ring structure. A moiety with two degrees of unsaturation may include two double bonds, one triple bond, two ring structures, one double bond and one ring structure, or one ring structure that includes one double bond. A double bond corresponds to one double bond equivalent. A triple bond corresponds to two double bond equivalents.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention. With respect to pharmaceutical compositions, the term “consisting essentially of” includes the active ingredients recited, excludes any other active ingredients, but does not exclude any pharmaceutical excipients or other components that are not therapeutically active.

The term “consisting of” refers to compositions, methods, and respective components thereof as described herein, which are exclusive of any element not recited in that description of the embodiment.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

### II. POLYMERS

Aspects of the present disclosure relate to various polymers. Polymers disclosed herein may be useful in the generation of nanoparticles. The disclosed polymers may include, for example, functionalized polymers. A functionalized polymer may be a pegylated polysaccharide or a monosaccaride poly(orthoester). In some embodiments, disclosed herein are functionalized poly(orthoester) polymers comprising one or more hydrophobic groups. Functionalized poly(orthoester)s of the present disclosure may be useful as a nanoparticle co-assembly agent to facilitate generation of a nanoparticle comprising, for example, an amphiphile.

In some embodiments, the present disclosure provides a polymer having the general formula:

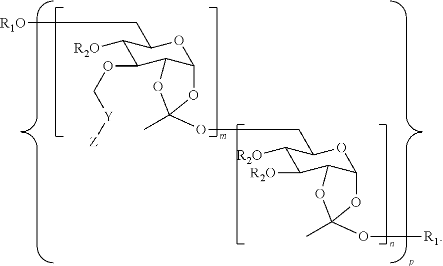

In some embodiments, R₁ is alkyl, acyl, or H, R₂ is alkyl, acyl, or H, m is an integer ranging from 1 to 10 (or any range derivable therein). In some embodiments, m is 1, 2, 3, 4, 5, 6, 7, 8, 9, or 10 (or any range derivable therein). In some embodiments, n is an integer ranging from 0 to 10 (or any range derivable therein). In some embodiments, n is 0, 1, 2, 3, 4, 5, 6, 7, 8, 9, or 10. In some embodiments, the ratio of m:n is 1:0, 1:1, 1:2, 1:3, 1:4, 1:5, 1:6, 1:7, 1:8, 1:9, 1:10, 2:1, 3:1, 3:2. 4:1. 4:2. 4:3. 5:1, 5:2, 5:3, 5:4, 6:1, 6:2, 6:3, 6:4, 6:5, 7:1, 7:2, 7:3, 7:4, 7:5, 7:6, 8:1, 8:2, 8:3, 8:4, 8:5, 8:6, 8:7, 9:1, 9:2, 9:3, 9:4, 9:5, 9:6, 9:7, 9:8, 10:1, 10:2, 10:3, 10:4, 10:5, 10:6, 10:7, 10:8, or 10:9. In some embodiments, the ratio of m:n is 1:0, 1:1, 1:5, or 1:10. In some embodiments, the ratio of m:n is 1:5.

In some embodiments, Y is a linker. Y may be any chemical linker. In some embodiments, Y includes a maleimide moiety, a PEG moiety, and/or a triazole moiety. In some aspects, the PEG moiety comprises from 2 to 20 ethyleneoxy units. In some embodiments, Y is a triazole linker. In some embodiments, Z is a hydrophobic group. Z may be an alkyl group or an acyl group. In some embodiments, Z has at least, at most, or exactly 2, 3, 4, 5, 6, 7, 8, 9, 10, 11, 12, 13, 14, 15, 16, 17, 18, 19, or 20 carbon atoms. In some embodiments, Z has at least 10 carbon atoms. In some embodiments, Z has 18 carbon atoms. In some aspects, Z includes at least one degree of unsaturation. In some embodiments, Z is an oleyl group. In some embodiments, p is an integer ranging from 2 to 500. In some embodiments, p is 2, 3, 4, 5, 6, 7, 8, 9, 10, 15, 20, 30, 40, 50, 100, 200, 300, 400, 500, or any range derivable therein.

In some embodiments, a polymer of the present disclosure has formula:

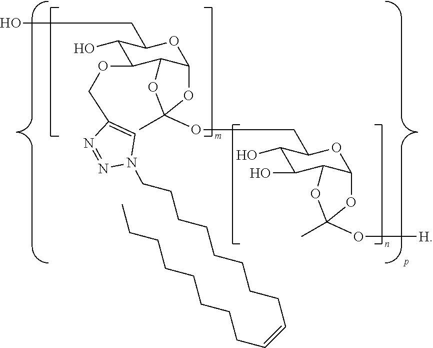

where the ratio of m:n is 1:5.

It is further contemplated that, in some embodiments, any one or more of these embodiments or elements may be excluded.

### III. NANOPARTICLES

Aspects of the disclosure are directed to nanoparticles, including self-assembled nanoparticles, useful for delivery of one or more agents. In some embodiments, disclosed herein are self-assembled nanoparticles comprising an amphiphile and a co-assembly polymer. The amphiphile may be or comprise an agent for delivery using the nanoparticle. For example, a nanoparticle may comprise an amphiphile comprising a therapeutic or diagnostic agent and a co-assembly polymer. Alternatively, the nanoparticle may comprise a hydrophobic agent. In some embodiments, such a nanoparticle is useful for delivery of the agent to an individual, for example, for therapeutic or diagnostic purposes, as described in further detail elsewhere herein. Example agents which may be delivered using the disclosed nanoparticles include immune stimulating agents (e.g., TLR agonists), chemotherapeutic agents (e.g., paclitaxel), and other therapeutic agents.

In some embodiments, disclosed herein is a nanoparticle comprising an amphiphile and a nanoparticle co-assembly agent. In some embodiments, an amphiphile comprises an amphiphilic group, a hydrophilic linker, and a hydrophobic group. An amphiphile may co-assemble with a nanoparticle co-assembly agent, thereby generating a nanoparticle. An amphiphile may comprise one or more agents for delivery. In some embodiments, an amphiphile comprises one or more PRR agonists (e.g., TLR agonists). In some embodiments, an amphiphile comprises two agents for delivery. In some embodiments, an amphiphile comprises an amphiphilic therapeutic agent and a hydrophobic therapeutic agent, where the therapeutic agents are attached by a hydrophobic linker. In one example, an amphiphile comprises linked TLR agonists.

In some embodiments, a nanoparticle co-assembly agent comprises a functionalized polymer. In some embodiments, a nanoparticle co-assembly agent is a functionalized hydrophilic polymer comprising a co-assembly agent linker and a hydrophobic group. Such a co-assembly agent may interact with an amphiphile via hydrophobic and hydrophilic interactions, thereby facilitating generation of a nanoparticle. In some embodiments, a nanoparticle co-assembly agent is a functionalized monosaccharide poly(orthoester). A monosaccharide poly(orthoester) may be a glucose poly(orthoester), which may be functionalized with one or more hydrophobic groups. A nanoparticle co-assembly agent may be a polymer of the present disclosure, as described in more detail elsewhere herein.

Also disclosed are methods of making nanoparticles of the present disclosure. Where a nanoparticle is a self-assembled nanoparticle, methods of making a nanoparticle may comprise providing the nanoparticle components in sufficient conditions for self-assembly. For example, methods of making a self-assembled nanoparticle may comprise providing an amphiphile and functionalized polymer of the present disclosure in conditions sufficient for self-assembly. In some embodiments, an amphiphile and a functionalized polymer are provided in a salt solution. In some embodiments, a salt solution is necessary for nanoparticle formation. In some embodiments, the salt solution is a balanced salt solution such as, for example, phosphate buffered saline (PBS). Methods for making nanoparticles may further comprise subjecting a solution to nanoprecipitation and/or dialysis, thereby facilitating nanoparticle formation.

### IV. PROTEINS AND POLYPEPTIDES

As used herein, a “protein” refers to a molecule comprising at least five amino acid residues. A “polypeptide” or “peptide,” as used herein, refers to a molecule comprising at least three amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein or polypeptide is specifically mentioned herein, it is in general a reference to a native (wild-type) or recombinant protein or, optionally, a protein in which any signal sequence has been removed. The protein or polypeptide may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

A peptide or polypeptide of the present disclosure may be naturally occurring or may be synthetic. A polypeptide of the disclosure may be at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length, or any range or value derivable therein. A polypeptide of the disclosure may be described based on its hydrophobicity and/or hydrophilicity. A hydrophilicity score of a polypeptide may be determined based on a hydrophilicity value of each component amino acid. U.S. Pat. No. 4,554,101, incorporated herein by reference, describes that the following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). As used herein, a “hydrophilic polypeptide” describes a polypeptide having an average hydrophilicity score greater than 0 based on the sum of the hydrophilicity values of all amino acid residues of the polypeptide.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

### V. PRR AGONISTS AND TLR AGONISTS

Certain aspects of the present disclosure are directed to pattern recognition receptor (PRR) agonists. A PRR agonist may be any molecule that, directly or indirectly, activates a PRR or stimulates PRR signaling. PRRs include cell surface receptors (e.g., toll-like receptor (TLR) agonists) and intracellular receptors (e.g., RIG-I-like receptors). Examples of PRRs targeted by agonists of the present disclosure include NOD-like receptors, RIG-I-like receptors, STING receptors, and toll-like receptors (TLRs). In some embodiments, disclosed herein are PRR agonists, wherein a PRR agonist is a NOD-like receptor agonist, a RIG-I-like receptor agonist, a STING agonist, or a TLR agonist. In some embodiments, a PRR agonist is a TLR agonist.

Aspects of the present disclosure relate to TLR agonists. A TLR agonist may be any molecule that, directly or indirectly, activates a TLR and/or stimulates TLR signaling. In some cases, a TLR agonist is a molecule that binds directly to a TLR. In some cases, TLR agonists of the present disclosure are linked, for example, by a polyethylene glycol (PEG) or other molecular linker. TLR agonists may be formulated into nanoparticles, optionally with one or more co-assembly agents (e.g., functionalized polymers).

In some embodiments, the TLR agonist is one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111:B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam₂CSK_{4;} lipoarabinomannan or lipomannan from M. smegmatis; triacylated lipoproteins such as Pam₃CSK₄; lipoproteins such as MALP-2 and MALP-404 from mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLRS (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™ Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CL097, 2Bxy, R848, or R848 VacciGrade™) TLR9 (e.g., CpG ODNs); and TLR11 (e.g., Toxoplasma gondii Profilin). In some embodiments, the TLR agonist is an amphiphilic TLR agonist. In some embodiments, the TLR agonist is a TLR 2/6 agonist, for example Pam₂CSK₄ or Pam₃CSK_{4.} In some embodiments, the TLR agonist is a hydrophobic TLR agonist. In some embodiments, the TLR agonist is a TLR 7, TLR 8, or TLR 7/8 agonist, for example 2Bxy. In certain embodiments, the TLR agonist is a specific agonist listed above. In further embodiments, the TLR agonist is one that agonizes either one TLR or two TLRs specifically. In some embodiments, linked TLR agonists comprise different types of TLR agonists (e.g., TLR agonists capable of activating different classes of TLRs). For example, a linked TLR agonist may comprise a TLR 2/6 agonist and a TLR 7 agonist covalently attached by a molecular linker. Alternatively, linked TLR agonists may comprise the same type of TLR agonist.

In some embodiments, disclosed herein are small molecule compounds suitable for use as TLR agonists. Examples of small molecule TLR agonists include compounds having a 2-aminopyridine fused to a five membered nitrogen-containing heterocyclic ring. Such compounds include, for example, imidazoquinoline amines including but not limited to substituted imidazoquinoline amines such as, for example, aminoalkyl-substituted imidazoquinoline amines, amide-substituted imidazoquinoline amines, sulfonamide-substituted imidazoquinoline amines, urea-substituted imidazoquinoline amines, aryl ether-substituted imidazoquinoline amines, heterocyclic ether-substituted imidazoquinoline amines, amido ether-substituted imidazoquinoline amines, sulfonamido ether-substituted imidazoquinoline amines, urea-substituted imidazoquinoline ethers, and thioether-substituted imidazoquinoline amines; tetrahydroimidazoquinoline amines including but not limited to amide-sub stituted tetrahydroimi dazoquinoline amines, sulfonamide-substituted tetrahydroimidazoquinoline amines, urea-substituted tetrahydroimidazoquinoline amines, aryl ether-substituted tetrahydroimidazoquinoline amines, heterocyclic ether-substituted tetrahydroimidazoquinoline amines, amido ether-substituted tetrahydroimidazoquinoline amines, sulfonamido ether-substituted tetrahydroimidazoquinoline amines, urea-substituted tetrahydroimidazoquinoline ethers, and thioether-substituted tetrahydroimidazoquinoline amines; imidazopyridine amines including but not limited to amide-substituted imidazopyridine amines, sulfonamido-substituted imidazopyridine amines, urea-substituted imidazopyridine amines; aryl ether-substituted imidazopyridine amines, heterocyclic ether-substituted imidazopyridine amines, amido ether-substituted imidazopyridine amines, sulfonamido ether-substituted imidazopyridine amines, urea-substituted imidazopyridine ethers, and thioether-substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines.

In certain embodiments, the TLR agonist is an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the TLR agonist is a sulfonamide-substituted imidazoquinoline amine. In alternative embodiments, the TLR agonist can be a urea-substituted imidazoquinoline ether. In another alternative embodiment, the TLR agonist can be an aminoalkyl-substituted imidazoquinoline amine. In one particular embodiment, the TLR agonist is 4-amino-a,a,2-trimethyl-1H-imidazo[4,5-c]quinolin-1-ethanol. In an alternative particular embodiment, the TLR agonist is N-(2-{2-[4-amino-2-(2-methoxyethyl)-1H-imidazo[4,5-c]quinolin-1-yl]ethoxy}ethyl)-N-methylmorpholine-4-carboxamide . In another alternative embodiment, the TLR agonist is 1-(2-amino-2-methylpropyl)-2-(ethoxymethyl)-1H-imidazo[4,5-c]quinolin-4-amine. In another alternative embodiment, the TLR agonist is N-[4-(4-an- no-2-ethyl-1H-imidazo[4,5-c]quinolin-1-yl)butyl]methanesulfonamide. In yet another alternative embodiment, the TLR agonist is N-[4-(4-amino-2-propyl-1H-imidazo[4,5-c]quinolin-1-yl)butyl]methanesulfonamide.

In certain embodiments, the TLR agonist may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an aminoalkyl-substituted imidazoquinoline amine, an amide-substituted imidazoquinoline amine, a sulfonamide-substituted imidazoquinoline amine, a urea-substituted imidazoquinoline amine, an aryl ether-substituted imidazoquinoline amine, a heterocyclic ether-substituted imidazoquinoline amine, an amido ether-substituted imidazoquinoline amine, a sulfonamido ether-substituted imidazoquinoline amine, a urea-substituted imidazoquinoline ether, or a thioether-substituted imidazoquinoline amines.

### VI. IMMUNOTHERAPY

A. Checkpoint Inhibitors and Combination Treatment

Embodiments of the disclosure may include administration of immune checkpoint inhibitors, which are further described below. Embodiments of the disclosure may include administration of one or more immune checkpoint inhibitors in combination with an additional therapeutic agent (e.g., TLR agonist(s)) described herein, nanoparticles described herein, etc.). For example, methods for treating cancer may comprise administration of an immune checkpoint inhibitor in combination with linked a TLR-agonist nanoparticle formulation of the present disclosure.

1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain complementarity determining regions (CDRs) or variable regions (VRs) of nivolumab, pembrolizumab, or pidilizumab. Accordingly, in one embodiment, the inhibitor comprises the CDR1, CDR2, and CDR3 domains of the heavy chain variable (VH) region of nivolumab, pembrolizumab, or pidilizumab, and the CDR1, CDR2 and CDR3 domains of the light chain variable (VL) region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or an oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Patent No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

3. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589 , WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

4. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM 032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range or value therein) variable region amino acid sequence identity with the above-mentioned antibodies.

B. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

C. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-C SF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

D. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

E. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNa and IFNβ), type II (IFNγ) and type III (IFNλ).

Interleukins have an array of immune system effects. IL-2 is an exemplary interleukin cytokine therapy.

F. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### VII. PHARMACEUTICAL COMPOSITIONS

The present disclosure includes methods for treating disease and modulating immune responses in a subject in need thereof. The disclosure includes nanoparticles (e.g., nanoparticles comprising linked TLR-agonists) that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 2, 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

### VIII. ADMINISTRATION OF THERAPEUTIC COMPOSITIONS

The therapy provided herein may comprise administration of a combination of therapeutic agents, such as a first therapy (e.g., immunotherapy, linked TLR agonists disclosed herein, nanoparticles disclosed herein comprising therapeutic agents, etc.) and a second therapy (e.g., chemotherapy, radiotherapy, etc.). The therapies may be administered in any suitable manner known in the art. For example, the first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, a cancer therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 25 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range or value derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IX. EXAMPLES

The following examples are included to demonstrate preferred embodiments of the disclosure. It should be appreciated by those of skill in the art that the techniques disclosed in the examples which follow represent techniques discovered to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. The Examples should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### Example 1

### Synthesis and Characterization of Compounds

### General Information

All reactions were conducted under dried nitrogen or argon stream. Anhydrous solvents (CH2Cl2 99.8%, benzene 99.8%) were purchased in capped DriSolv™ bottles, used without further purification, and stored under argon. All other solvents and reagents were used without further purification. All glassware utilized were flame-dried before use. Glass-backed TLC plates (Silica Gel 60 with a 254 nm fluorescent indicator) were used without further manipulation and stored over desiccant. Developed TLC plates were visualized under a short-wave UV lamp, and/or by heating plates that were dipped in ammonium molybdate/cerium (IV) sulfate solution. Silica gel column chromatography was performed using flash silica gel (32-63 μm) and employed a solvent with polarity correlated with the TLC mobility.

IR spectra were obtained on a Perkin Elmer Spectrum BX Fourier-transform infrared (FTIR) spectrometer using NaCl plates, with the sample being deposited from CH2Cl2 and allowing for evaporation of the solvent under ambient temperature.

Mass spectrometry was measured with a Waters LCT Premier™ XE unit.

¹H NMR spectra were recorded at 300 MHz on a Varian Mercury 300 or 500 MHz on a Varian Inova 500 spectrometer or a Bruker 500 spectrometer, with tetramethylsilane (TMS) proton signal as the standard. 13C NMR spectra were recorded at 126 MHz on a Varian Inova 500 spectrometer, with tetramethylsilane (TMS) carbon signal as the standard.

Gel permeation chromatography (GPC) analyses were conducted using a Viscotek GPC system equipped with a VE 3580 RI detector, VE 112 solvent delivery system, and a column system comprised of one PAS102 and one PAS103 column (Polyanalytik Inc.). The system was equilibrated at 35° C. in THF, which served as the polymer solvent and eluent with a flow rate of 1.0 mL min-1. Polymer solutions were prepared at a known concentration (ca. 6 mg/mL) and an injection volume of 100 μL was used. Data collection and analyses were performed by OmniSEC software system from Malvern Inc. The GPC system was calibrated using polystyrene standards having molecular weights of 2.5 5.0, 9.0, 17.0 and 50.0 kDa and PDI of 1.05-1.07 (Supelco Analytical, Bellefonte, Pa., USA).

### 1. Synthesis of Toll Like Receptor Agonist Heterodimer (2/6-7a)

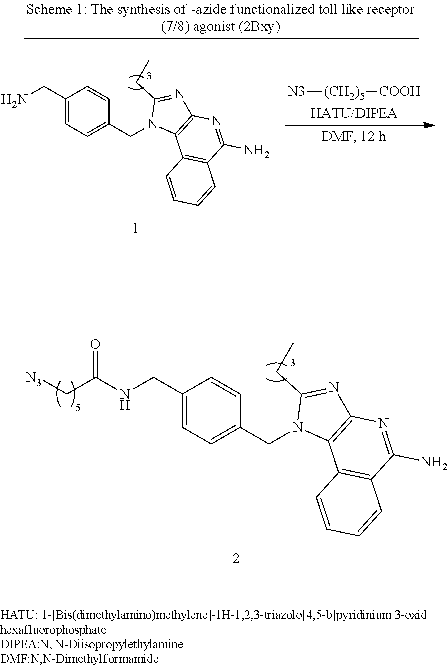

### 1.1 Synthesis of Toll Like Receptor Agonist (7/8a) azide (2)

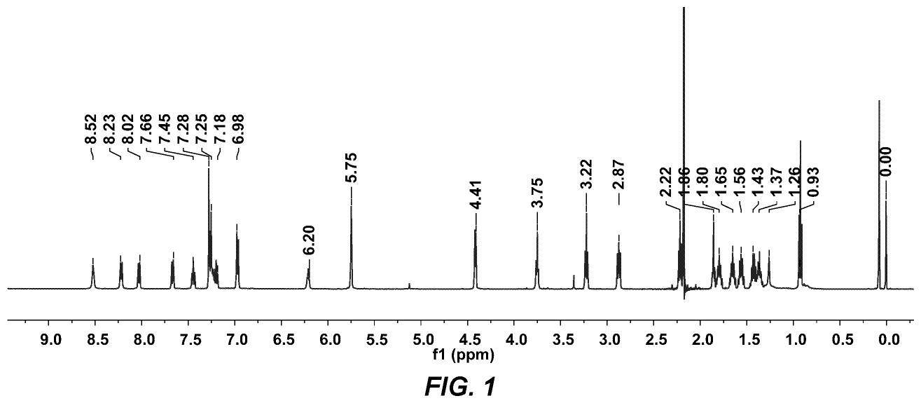

The synthesis of compound 1 was performed based on previously reported synthetic protocol (Albin, T. J. et al., ACS Cent. Sci. 2019). Compound 1 (100 mg, 0.28 mmol) and azidohexanoic acid (48.41 mg, 0.31 mmol) were added in anhydrous DMF following which DIPEA (73.2 μL, 0.42 mmol and HATU (159.70 mg,0.42 mmol) were added to the reaction mixture under argon. The reaction mixture was stirred for 12 h at room temperature following which the solvent was removed under vacuo and the product was purified by silica gel column chromatography using a gradient of 5% methanol in dichromethane, giving compound 2 as a yellow powder (104.8 mg, 0.21 mmol, 75% yield). ¹H NMR (500 MHz, CDCl₃) δ8.52 (s, 1H), 8.22 (d, J=8.3 Hz, 1H), 8.03 (d, J=8.3 Hz, 1H), 7.67 (d, J=8.3 Hz, 1H), 7.44 (t, J=8.3 Hz, 1H), 7.25 (d, J=8.1 Hz, 2H), 7.20 (m, 1H), 6.97 (d, J=8.1 Hz, 2H), 6.21 (s, 1H), 5.75 (s, 2H), 4.41 (d, 2H), 3.75 (m, 2H), 3.22 (t, 2H), 2.87 (t, 2H), 2.22 (m, 2H), 1.86 (s, 2H), 1.80 (m, 2H), 1.65 (m, 2H), 1.56 (m, 2H), 1.42 (m, 2H), 1.38-1.33 (m, 2H), 1.26 (s, 1H) 0.93 (t, 3H). ESI-MS: m/z calculated for C₂₈H₃₄₊₁N₈O: 499.64, observed: 499.65 (FIG. 1).

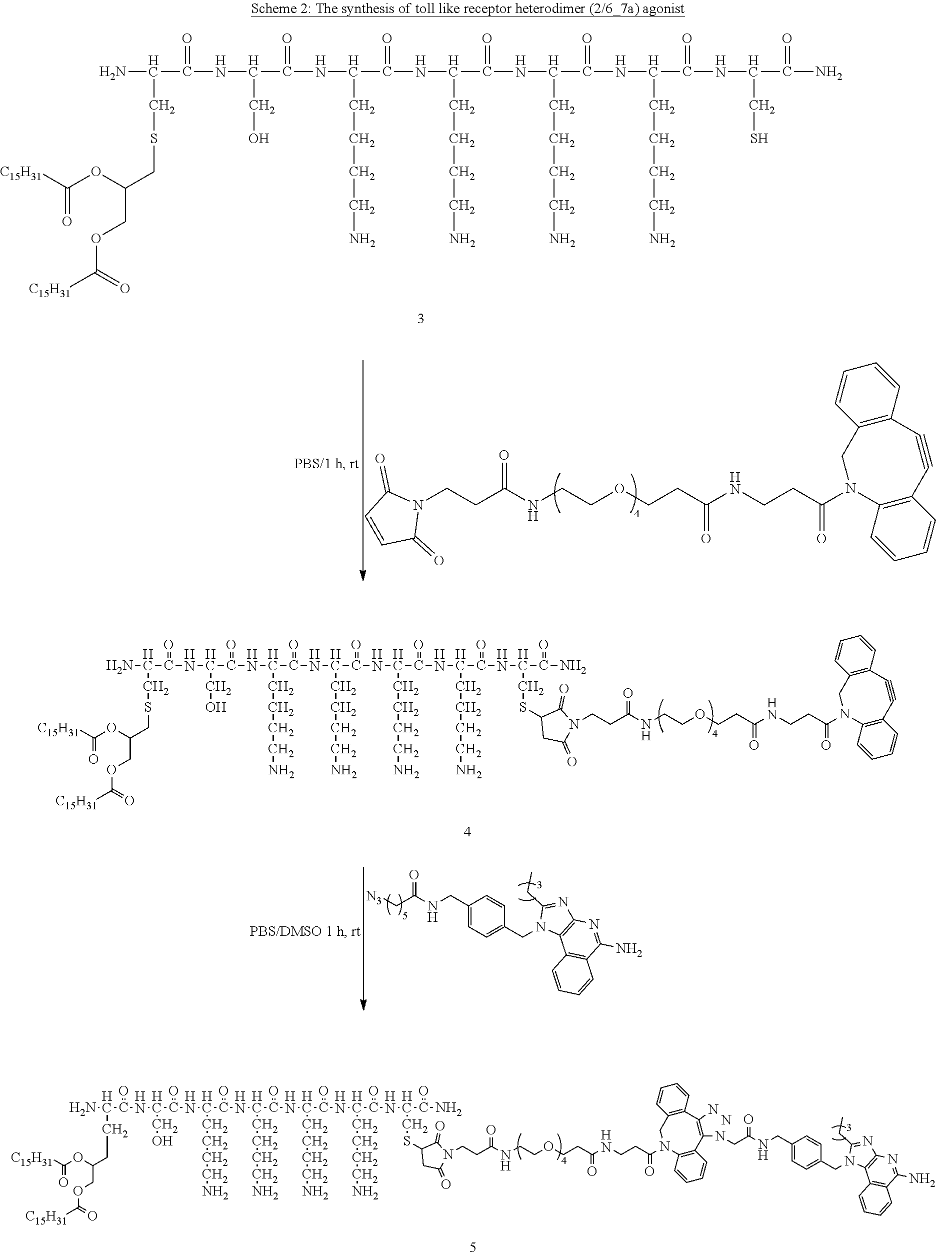

### 1.2 Synthesis of Toll Like Receptor 2/6 Agonist (Pam₂CSK₄C, 3)

Pam₂CSK₄C was synthesized employing solid phase peptide synthesis using Rink-amide resin on a CEM Liberty Blue automated microwave peptide synthesizer. Post synthesis, the peptide was deprotected using a cocktail of 85% TFA, 5% water, 5% anisole, 5% thioanisole. The crude peptide was precipitated in cold ether and further purified by reverse-phase HPLC using a C8 preparatory column, where the solvent system was A: water +0.1% TFA, B: acetonitrile +0.1% TFA (65-80% B over 15 mins). The HPLC fractions were lyophilized to afford the desired product as a white powder. MALDI-TOF m/z calculated for C₆₈H₁₃₂₊₁N₁₂O₁₂S₂: 1375.00, observed: 1375.01.

### 1.3 Synthesis of Compound (Pam₂CSK₄C-PEG₄-DBCO, 4)

Compound 3 (30 mg, 0.022 mmol) was dissolved in 1.5 mL PBS and added to a solution of maleimide-peg4-DBCO (14.84 mg, 0.022 mmol) in 1 ml PBS. The reaction mixture was stirred for 1 h after which it was purified by reverse-phase HPLC using a C8 preparatory column, where the solvent system was A: water +0.1% TFA, B: acetonitrile +0.1% TFA (65-80% B over 15 mins). The HPLC fractions were lyophilized to afford the desired product as a white powder. (Recovered: 29 mg, 0.014 mmol, 64% yield), MALDI-TOF m/z calculated for C₁₀₄H₁₇₄₊₁N₁₆O₂₁S₂: 2049.75, observed: 2049.79.

### 1.4 Synthesis of TLR 2/6_7/8 Agonist Heterodimer (Pam₂CSK₄C-PEG4-DBCO-2Bxy, 5)

Compound 4 (20 mg, 0.010 mmol) was dissolved in 1.5 mL PBS and added to a solution of compound 2 (5.5 mg, 0.011 mmol) in 1.5 mL DMSO. The reaction mixture was stirred for 1 h after which it was purified by reverse-phase HPLC using a C8 preparatory column, where the solvent system was A: water +0.1% TFA, B: acetonitrile +0.1% TFA (30-100% B over 15 mins). The HPLC fractions were lyophilized to afford the desired product as a white powder. (Recovered: 12.5 mg, 0.0049 mmol 49% yield), MALDI-TOF m/z calculated for C₁₃₂H₂₀₈₊₁N₁₆O₂₁S₂: 2548.39, observed 2549.41.

### 2. Synthesis of oleyl azide C₁₈H₃₅Br (Octadec-9-en-1-azide)

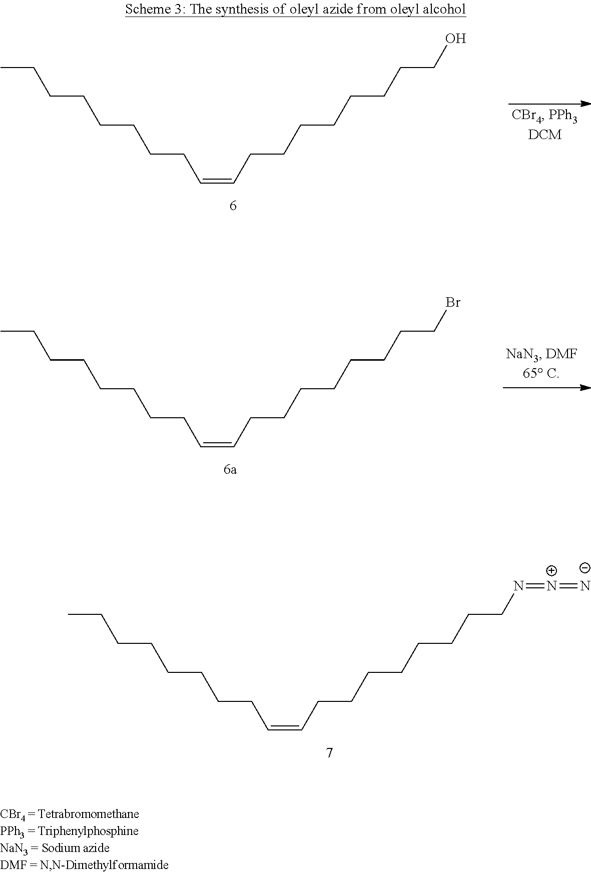 [SMILES: CCCCCCCC/C=C\CCCCCCCCO ; CCCCCCCC/C=C\CCCCCCCCBr ; CCCCCCCC/C=C\CCCCCCCCN=[N+]=[N-]]

### 2.1 Synthesis of oleyl bromide: C₁₈H₃₅Br (Octadec-9-en-1-Bromide, 6a)

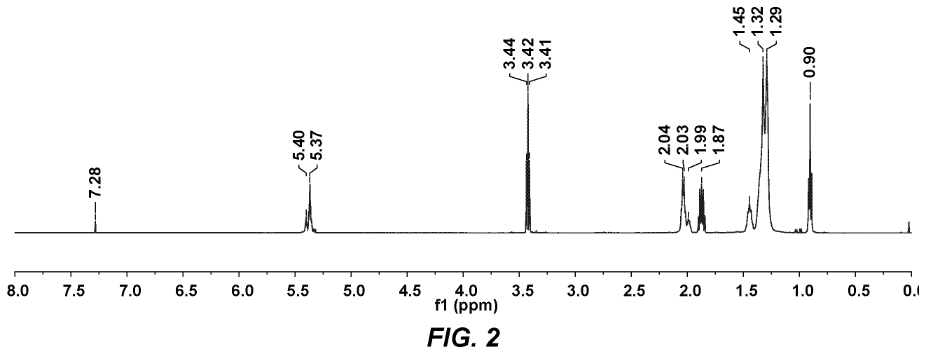

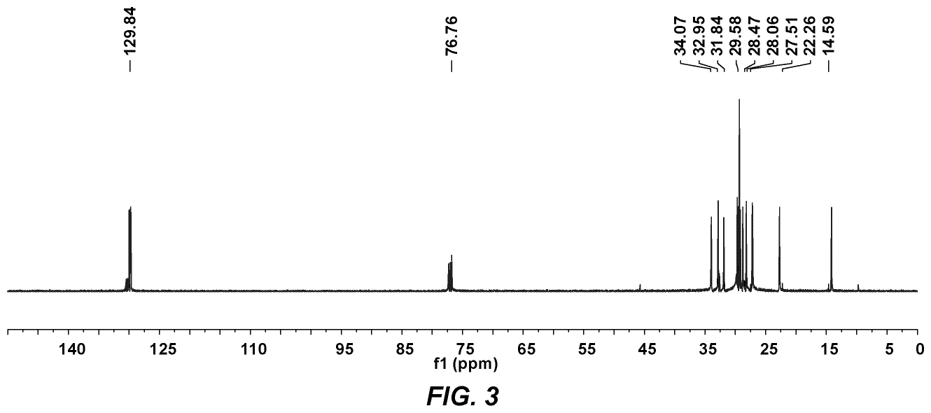

To a solution of 6 (1 g, 3.72 mmol) in anhydrous dichloromethane (40 mL) was added triphenylphosphine (1.5 g, 5.6 mmol) and tetrabromomethane (1.8 g, 5.6 mmol). The reaction mixture was stirred under nitrogen at rt for 16 h. After extraction, the solvent was removed under reduced pressure and the product was purified by silica gel column chromatography (hexanes/ethyl acetate =8/2, v/v, Rf =0.7) to afford the product 55% from 6. ¹H NMR (500 MHz, cdcl₃) δ5.38 (m, 2H vinyl), 3.42 (s, 2H (α-methylene to bromide), 2.02 (m, 4H, allylic), 1.92-1.84 (m, 2H, β-methylene to bromide), 1.44 (broad m, broad multiplet, 2H, γ-methylene to bromide), 1.38-1.26 (m, 20H, the remaining methylene groups),), 0.90 (t, 3H, terminal methyl group) (FIG. 2).¹³C NMR (126 MHz, cdcl₃) δ129.84, 76.76, 34.07, 32.95, 31.84, 29.58, 28.47, 28.06, 27.51, 22.26, 14.59 (FIG. 3).

### 2.2 Synthesis of oleyl azide: C₁₈H₃₅N3 (Octadec-9-en-1-azide, 7)

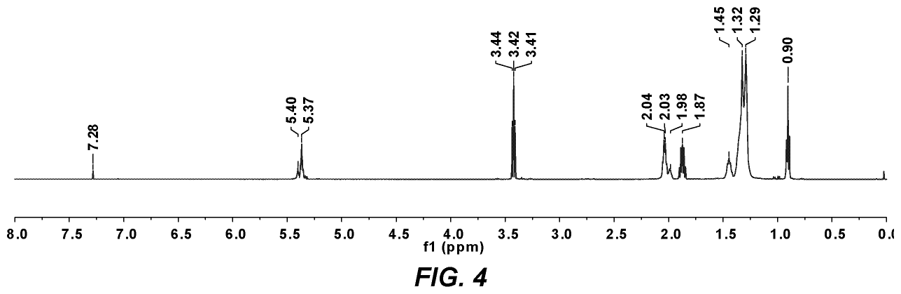

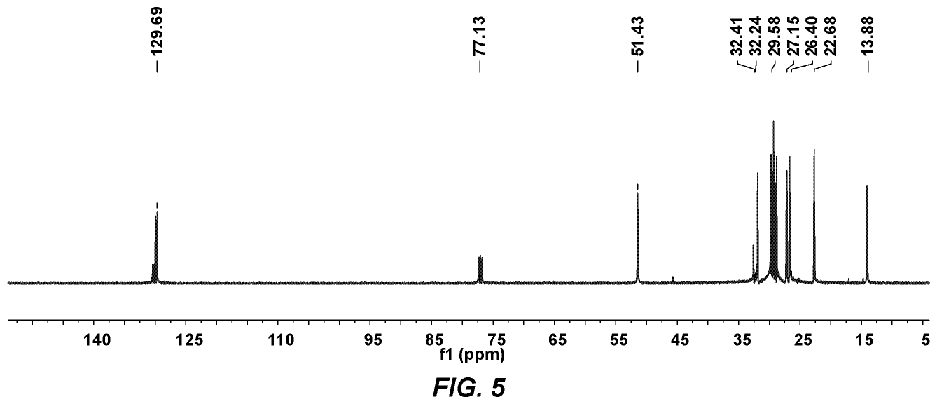

To a solution of 6a (1 g, 3.03 mmol) in anhydrous N,N dimethyl formamide (40 mL) was added sodium azide (0.4 g, 6.05 mmol) and tetra-n-butylammonium bromide (0.09 g, 0.030 mmol). The reaction mixture was refluxed at 65° C. for 16 h. The product 7 was purified by silica gel column chromatography ¹H NMR (500 MHz, cdcl3) δ5.37 (m, 2H vinyl), 3.26 (t, 2H α-methylene to azide), 2.03 (m, 4H allylic), 1.98 (m, 2H β-methylene to azide), 1.66-1.55 (m, 2H, γ-methylene to azide), 1.33 (m, 20H, the remaining methylene groups), 0.89 (t, 3H, terminal methyl group) (FIG. 4). 13C NMR (126 MHz, cdcl3) δ129.69, 77.13, 51.43, 32.41, 32.24, 29.58, 27.15, 26.40, 22.68, 13.88 (FIG. 5).

### 3. Synthesis of Deprotected oleyl Grafted Sugar Poly(orthoesters) (OL-DSPOE)

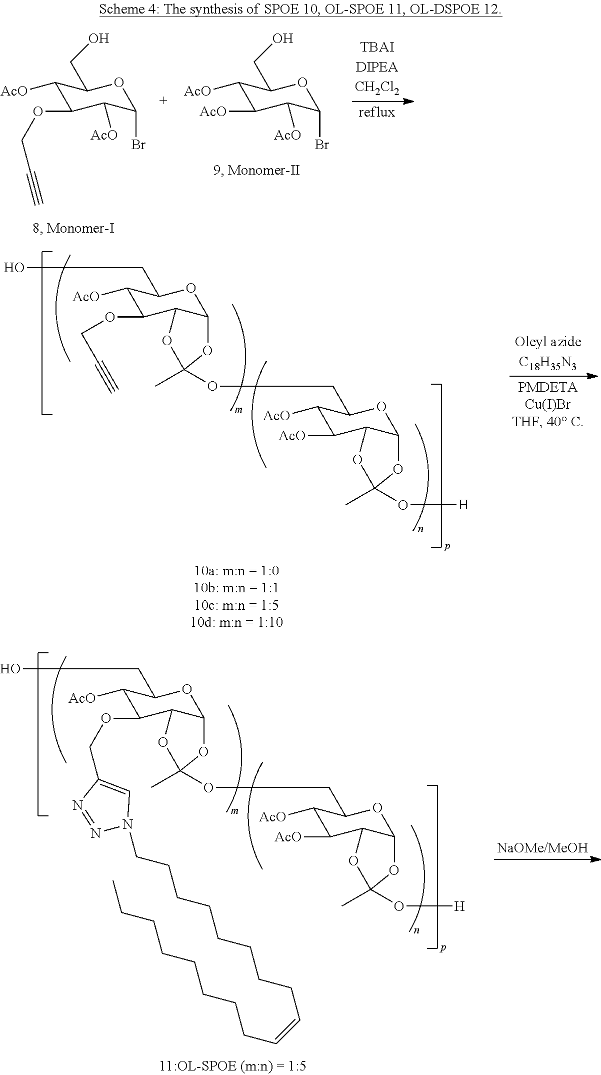

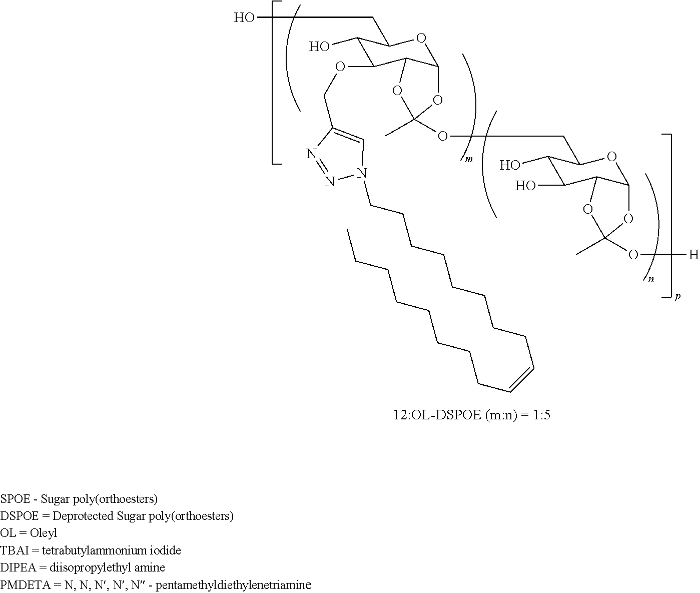

### 3.1 Synthesis of 3-propargyl-2,4-di-O-acetyl-α-D-glucopyranosyl bromide (monomer I) (8)

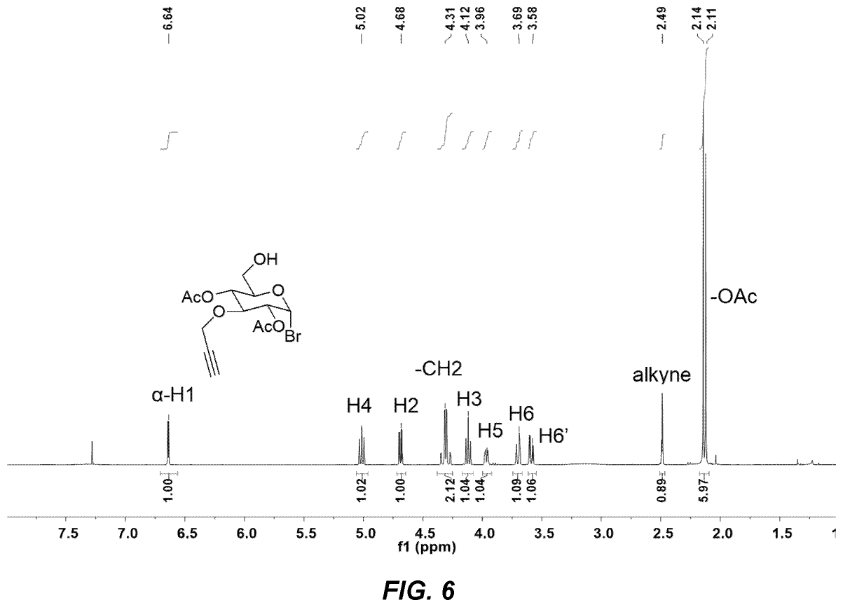

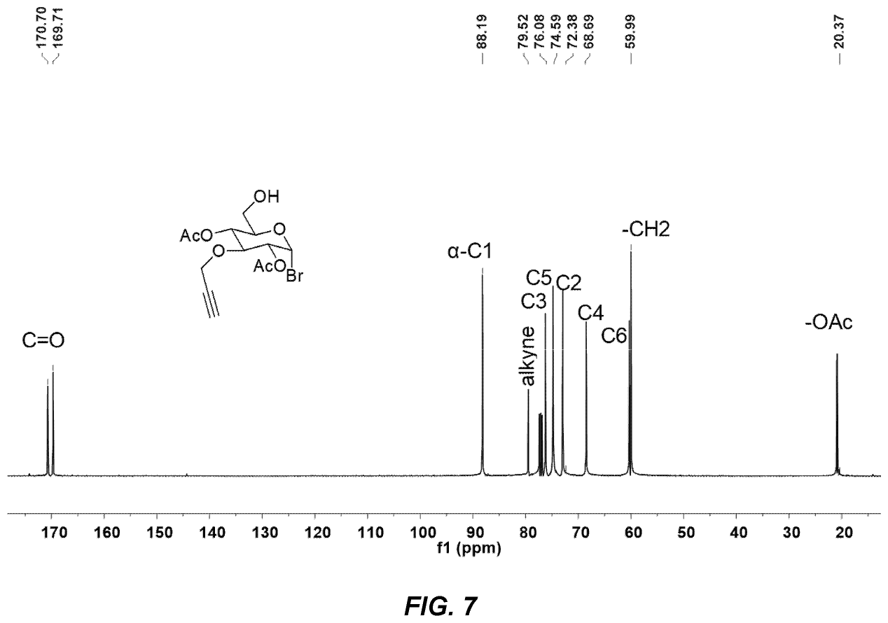

The synthesis of compound 8 was performed based on previously reported synthetic protocol (Maiti, S. et al., J. Am. Chem. Soc. 2019). Briefly, in a 50 mL disposable polypropelene tube, 3-propargyl-2,4-di-O-acetyl-6-O-tert-butyldiphenylsilyl-α-D-glucopyranosyl bromide (1.0 g, 1.7 mmol) was added to anhydrous tetrahydrofuran (7.0 mL). HF-Py (0.82 g, 70% HF in pyridine, 0.57 g HF, 29 mmol HF) was subsequently added. The reaction was stirred under nitrogen at 4° C. for 12 h. The solvent and excess HF-Py was removed by sparging N2 gas at 4° C. and the residue was purified by silica gel column chromatography (hexanes/ethyl acetate=6/4, R_{f}=0.3) to afford the product as a white powder (0.40 g, 66%). ¹H NMR (500 MHz, CDCl₃) δ6.64 (d, J=3.9 Hz, 1H, a-H1), 5.02 (m, 1H, H-4), 4.68 (dd, J=9.6, 3.9 Hz, 1H, H-2), 4.31 (m, 2H, —CH₂), 4.12 (dd, J2,3 =10 Hz, J_{3,4}=10 Hz, 1H, H-3), 3.96 (m, 1H, H-5), 3.69 (ddd, J_{6,6′}=13.2 Hz, J_{6,OH=}8.2 Hz, J_{5,6}=2.2 Hz, 1H, H-6), 3.58 (ddd, J_{6,6′}=13.2 Hz, J_{6′,OH}=6.2 Hz, J_{5,6}=3.5 Hz, 1H, H-6′), 2.49 (t, J=2.4 Hz, 1H, HC≡C), 2.13 (d, J=10.0 Hz, 6H),), 2.14 (s, 3H), 2.11 (s, 3H) (FIG. 6). ¹³C NMR (126 MHz, CDCl₃) δ170.66 (C═O), 169.27 (C═O), 88.47 (α-C1), 79.54 (acetylene —CH), 76.33 (C-3), 74.77 (C-5), 72.73 (C-2), 68.69 (C-4),61.08 (O—CH₂), 60.31 (C-6), 20.86 (—OAc) (FIG. 7). ESI-MS calc for C₁₃H₁₇BrNaO₇ [M+Na]⁺=388.17, found: 388.06.

### 3.2 Synthesis of 2,3,4-tri-O-acetyl-α-D-glucopyranosyl bromide (monomer II) (9)

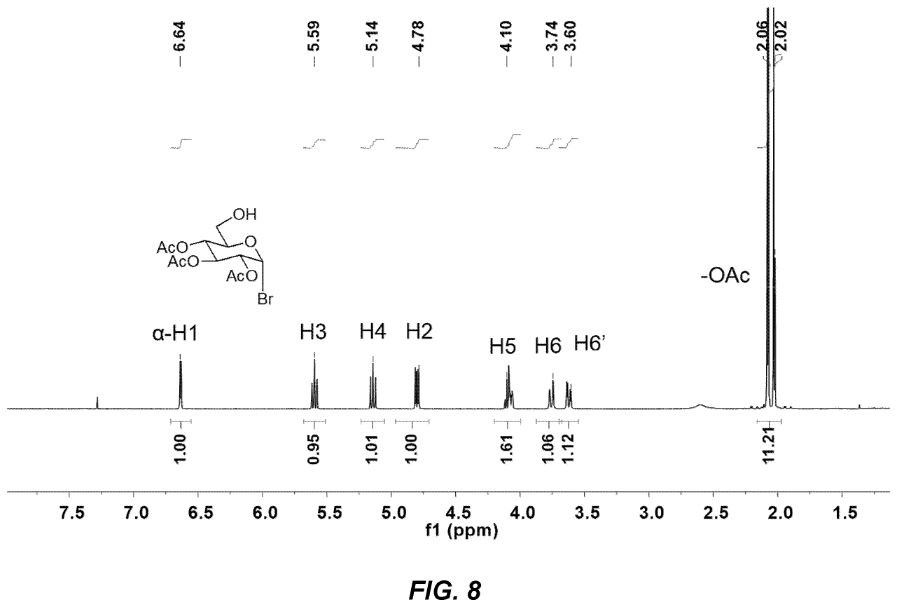

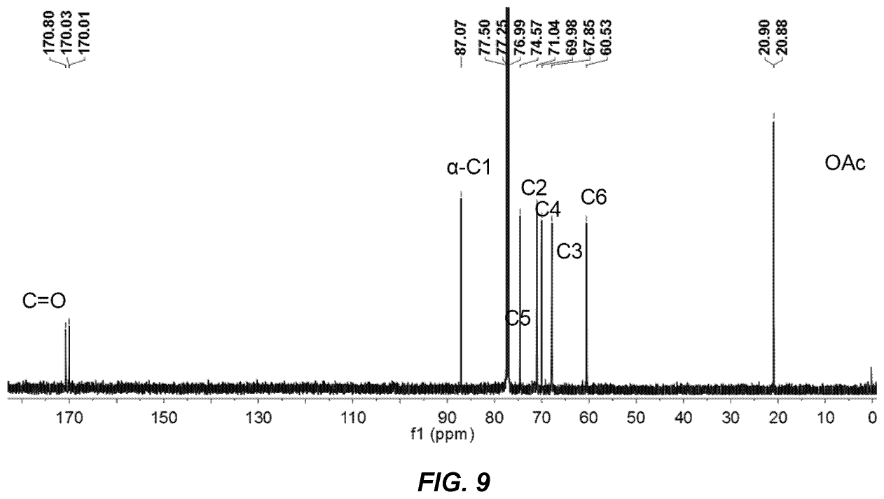

The synthesis of compound 9 was performed based on previously reported synthetic protocol (Maiti, S. et al., J. Am. Chem. Soc. 2019). Briefly, in a 50 mL disposable polypropelene tube, 2,3,4-tri-O-acetyl-6-O-tert-butyldiphenylsilyl-α-D-glucopyranosyl bromide (1.0 g, 1.65 mmol) was added to anhydrous tetrahydrofuran (7 mL). HF-Py (0.82 g, 70% HF in pyridine, 0.57 g HF, 29 mmol HF) was subsequently added. The reaction was stirred under nitrogen at 4° C. for 12 h. The solvent and excess HF-Py was removed by sparging N₂ gas at 4° C. and the residue was purified by silica gel column chromatography (hexanes/ethyl acetate=6/4, R_{f}=0.3) to afford the product as a white powder (0.38 g, 62%). ¹H NMR (300 MHz, CDCl₃) δ6.64 (d, J_{1,2}=4.0 Hz, 1H, α-H1), 5.63 (dd, J_{2,3}=10 Hz, J_{3,4}=10 Hz, 1H, H-3), 5.14 (dd, J_{3,4}=10 Hz, J_{4,5}=10 Hz, 1H, H-4), 4.80 (dd, J_{2,3}=10 Hz, J_{1,2}=4.0 Hz, 1H, H-2), 4.09-4.06 (m, 1H, H-5), 3.78 (ddd, J_{6,6′}=13.2 Hz, J_{6,OH}=8.2 Hz, J_{5,6}=2.2 Hz, 1H, H-6), 3.63 (ddd, J_{6,6′}=13.2 Hz, J_{6′,OH}=6.2 Hz, J_{5,6}=3.5 Hz, 1H, H-6′), 2.29 (dd, J_{6,OH}=8.2 Hz, J_{6′,OH}=6.2 Hz, 1H, —OH), 2.10 (s, 3H), 2.09 (s, 3H), 2.05 (s, 3H) (FIG. 8). ¹³C NMR (126 MHz, CDCl₃) δ170.80 (C═O), 170.03 (C═O), 170.01 (C═O), 87.07 (α-C1), 74.57 (C-5), 71.04 (C-2), 69.98 (C-3), 67.85 (C-4), 60.53 (C-6), 20.90 (—OAc), 20.88 (—OAc) (FIG. 9). ESI-MS calc for C₁₂H₁₇BrNaO₈ [M+Na]⁺=391.00, 393.00, found: 391.12, 393.13.

### 3.3 Synthesis of Fnctional Glucose Co-Poly(orthoester)(SPOE, 10)

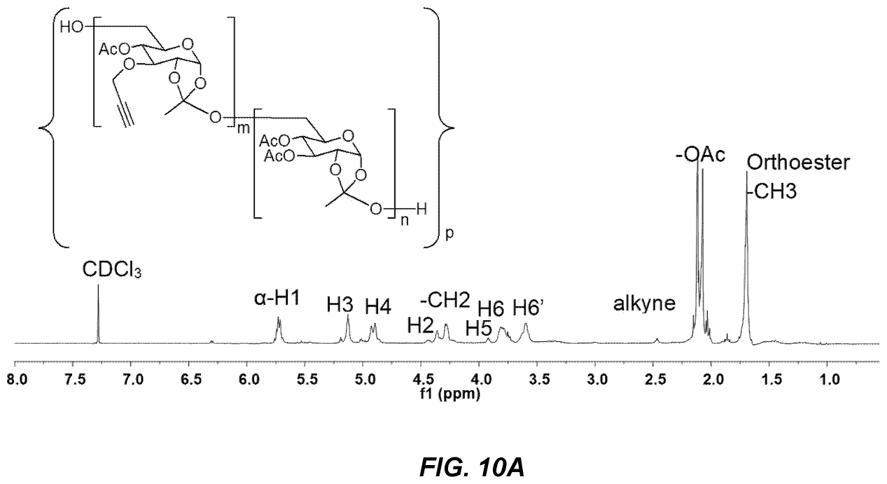

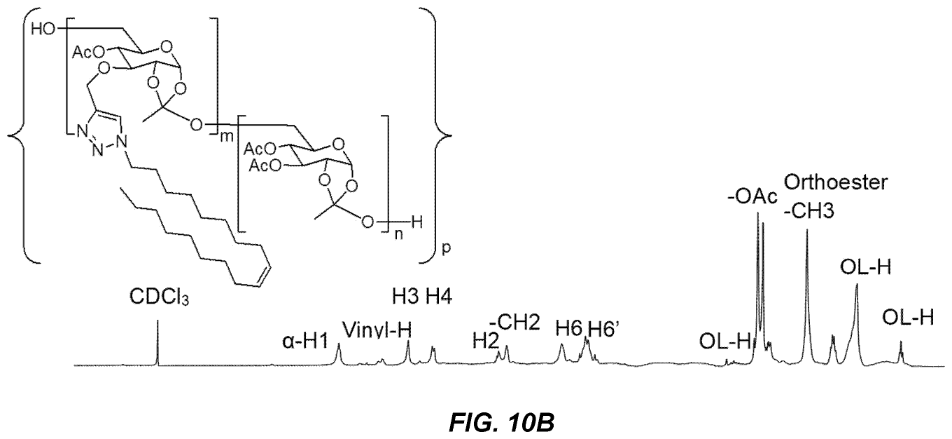

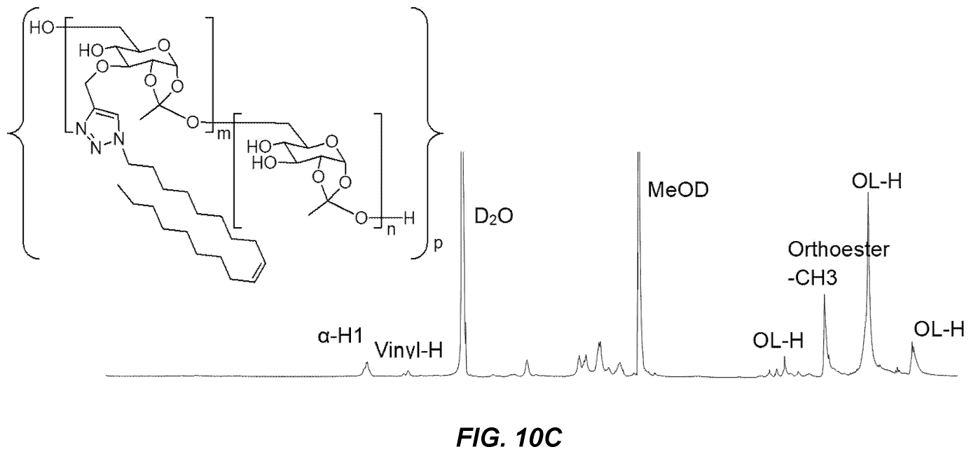

To a Schlenk flask was added monomer I (0.20 g, 0.54 mmol), monomer II, (0.20 g, 0.54 mmol), anhydrous CH₂Cl₂ (5 mL), tetrabutylammonium iodide (TBAI) (0.199 g, 0.54 mmol) and N,N-diisopropylethylamine (DIPEA, 0.21 g, 1.63 mmol). The reaction mixture was refluxed under argon atmosphere for 20 h. The solvent was removed by reduced pressure. The polymer was precipitated three times using a mixture of water/methanol (9/1, v/v) at 4° C. to afford 10 C as a white powder (0.22 g, 70%). Mₙ^{GPC}=6.3 kDa, PDI=1.3. ¹H NMR (500 MHz, CDCl₃) δ=5.71 (d, J=5.0, 1H, α-H1), 5.13 (m, 1H, H-3), 4.92-4.90 (t, J=9.0, 1H, H-4), 4.42 (s, —CH₂), 4.27 (m, 1H, H-2), 3.80_3.78 (m, 1H, H-5), 3.62-3.55 (m, 2H, H-6,6′), 2.44 (t, acetylene proton), 2.11 (m, 3H), 2.07 (s, 3H), 1.69 (s, 3H). ¹³C NMR (126 MHz, CDCl3) 169.99 (C═O), 169.51 (C═O), 121.34 (orthoester C), 97.30 (C1), 80.06 HCC), 72.81 (C-2), 70.09 (C-3), 68.35 (C-4), 67.81 (C-5), 63.25 (C-6), 21.12 (—OAc), 20.68 (—OAc), 20.59 (—CH₃) (FIG. 10A).

### 3.4 Synthesis of oleyl-grafted polysaccharide (OL-SPOE, 11)

To a flame dried 10-mL Schlenk flask was added co-polymer (SPOE) 10 (0.037 g, Mₙ^{GPC}=6.3 kDa, 0.021 mmol alkynes) and oleyl azide (0.074 g, 0.025 mmol). Anhydrous THF (3.0 mL) was added. After three cycles of freeze-pump-thaw, Cu(I)Br (1.0 mg, 0.0063 mmol) and N,N,N′,N′,N″-pentamethyldiethylenetriamine (4.73 mg, 0.0273 mmol) were added. The reaction was stirred at 40° C. for 12 h. The reaction mixture was loaded into a short column of neutral alumina to remove the copper catalyst. The reaction mixture was further precipitated in diethyl ether (3×10 mL) to afford the product (OL-SPOE, 11) as a very light brown powder (0.035 g, 80%). Mₙ^{GPC}=7.4 kDa; PDI=1.3. ¹H-NMR (500 MHz, CDCl₃) δ=5.73 (m, 1H, H-1), 5.34 (m, 2H vinyl), 5.12 (m, 1H, H-3), 4.90 (m, 1H, H-4), 4.34 (m, 2H, —CH₂), 4.27 (m, 1H, H-2), 3.79 (m, 1H, H-5), 3.62-3.55 (m, 2H, H-6,6′), 3.42 (s, 2H (α-methylene to bromide), 2.11-2.07 (s, 9H acetate), 2.03 (m, 4H allylic), 1.98 (m, 2H β-methylene) 1.69 (s, 3H orthoesters), 1.33 (m, 20H, the remaining methylene groups), 0.89 (t, 3H, terminal methyl group) (FIG. 10B).

### 3.5 Synthesis of deprotected oleyl-grafted polysaccharide (OL-DSPOE, 12)

To a flame dried 10-mL Schlenk flask was added co-polymer (OL-SPOE) 11 (0.050 g, Mₙ^{GPC}=7.4 kDa, 0.0068 mmol). Anhydrous sodium methoxide solution (2-3 mL, 0.3 (M) was added. The reaction mixture was stirried for 6 h at room temperature. The reaction mixture was further precipitated in ethanol to afford the product as a light brown powder (0.016 g, 50%). Mₙ^{GPC}=4.8 kDa; PDI=1.3. ¹H-NMR (500 MHz, CDCl₃) δ=5.71 (m, 1H, H-1), 5.33 (m, 2H vinyl), 4.29-3.47 (remaining H from glucose), 3.42 (s, 2H (α-methylene to bromide), 2.03 (m, 4H allylic), 1.98 (m, 2H β-methylene) 1.69 (s, 3H orthoesters), 1.33 (m, 20H, the remaining methylene groups), 0.89 (t, 3H, terminal methyl group) (FIG. 10C). A deprotected oleyl-grafted polysaccharide, e.g., compound 12, may be further reacted to convert the saccharide hydroxyl groups into ether groups (by conjugation to an alkyl group) or ester groups (by conjugation to an acyl group).

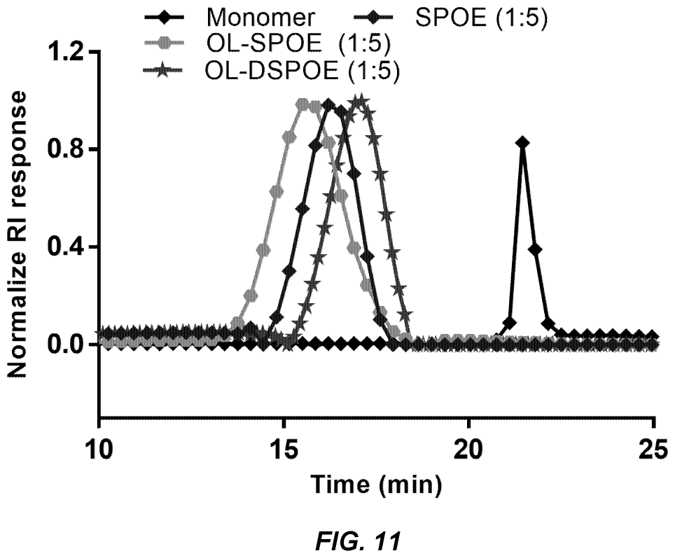

FIG. 11 shows results from gel permeation chromatography (GPC) analysis of SPOE (10), OL-SPOE (11), and OL-DSPOE (12).

### Example 2

### Nanoparticle Generation

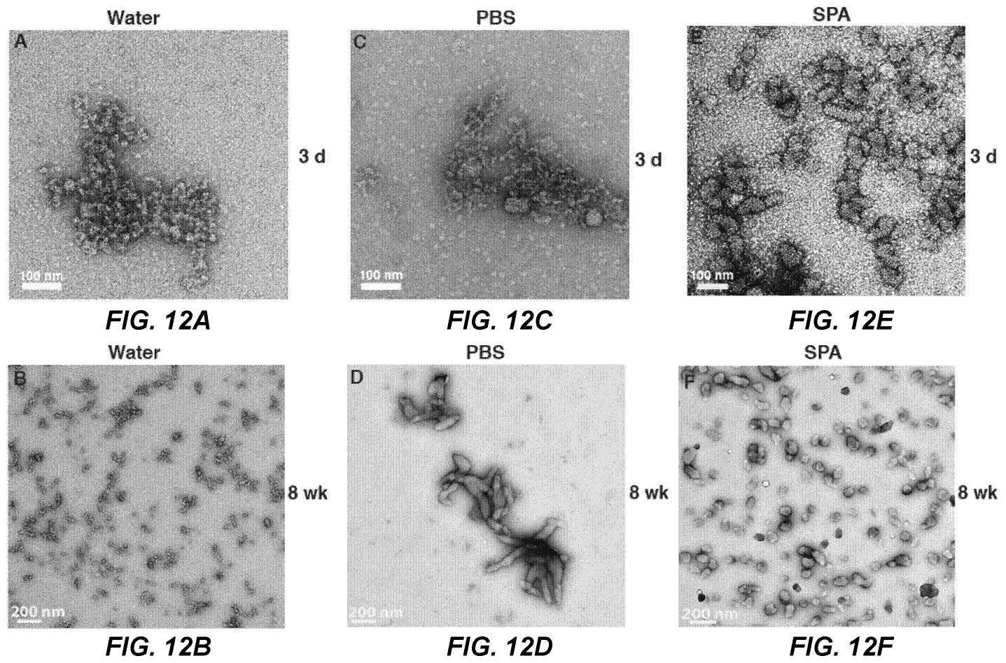

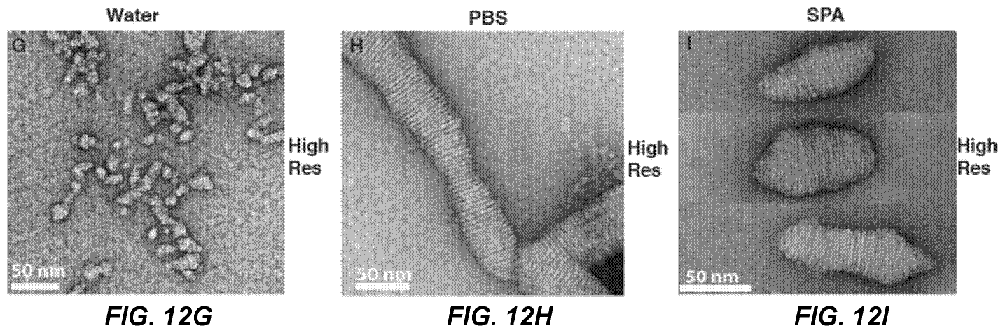

To test the efficacy of linked TLR agonists in cancer immunotherapy, a TLR agonist heterodimer was synthesized as described in Example 1, a where TLR 2/6 agonist (Pam₂CSK₄) was conjugated to an azide-functionalized TLR 7/8 agonist (2Bxy). The heterodimer amphiphile was designed having a hydrophilic peptide and short chain PEG segment flanked by hydrophobic units. Flexible linkers were appended between the 2/6 and 7/8 agonist segments for conformational freedom alongside with amphiphilic properties.²⁹ Typically, the combination of immiscible components in an amphiphile architecture results in formation of multicompartment micelles in aqueous solution.^{27,30} However, self-assembly of the heterodimer in water resulted in the formation of mostly random coiled structure (FIG. 12, panel A); which uncontrollably assembled into highly polydisperse random segmented fiber-like structures after 8 weeks at 4° C. (FIG. 12, panels B and G). Unlike in water, the heterodimer assembled in PBS with random coiled structures, with a small fraction forming self-assembled particles (FIG. 12, panel C). Over a period of eight weeks at 4° C., this assembled into large segmented worm-like micelles (FIG. 12, panel D and H). One possible explanation for this nucleation, coalescence and growth is a relatively short hydrophilic segment (SKC4-peg4) unable to shield the hydrophobic units (Pam2 and 2Bxy) resulting in several micellar units stacked up to share a common hydrophilic moiety. This indicates that further stabilization would be necessary to generate defined stable particles with faster kinetics. A molecular surfactant was synthesized containing a hydrophilic glucose poly(orthoester)³¹ grafted with hydrophobic oleyl units (synthesized as described in Example 1). The grafted sugar polymer readily assembled into defined nano-micelles (28.9±2.3 nm, ζ=−18.2±1.9 mV). It was hypothesized that the carbohydrate polymer would stabilize the TLR hetero-dimer nano-emulsion via hydrogen-bonding due to multiple OH-groups, electrostatic stabilization by negative zeta potential and hydrophobic stabilization via oleyl grafts.

To test the hypothesis, the sugar scaffold was admixed with the peptide amphiphile in a clinically relevant concentration. TEM analysis demonstrated that tuning the mass fraction of the sugar scaffold controlled the assembly into defined particles. An optimized formulation was obtained with 0.5:1 molar ratio of sugar and peptide resulting in the formation of well-defined particles within three days (FIG. 12, panel E) which were observed to be stable when stored at 4° C. for 8 weeks in PBS with no significant changes (FIG. 12, panels F and I). The length of majority of particles ranged from 50-75 nm and their width varied from 30-45 nm.

This falls within the range of particles capable of draining to tumor-sentinel lymph nodes (20-200 nm).^{32,33} The ζ potential of the particles were measured to be +(3.21±0.7) mV.

It was noted that introduction of the sugar scaffold induced a difference in surface pattern of the particles. (FIG. 12, panels H and I). Analysis of electron micrographs of the particles obtained from PBS sample indicated a consistent lamellar width with defined surface morphology, a contrast to lamella of the sugar-assisted particles that were found to have different width and pattern. CD analysis also indicated the generation of an ordered structure with a peak-shift (212 nm) possibly indicating the deposition of a stacked peptide β-sheet type structure to generate each lamella (supporting information). This novel class of material was named self-formulating PRR agonist (SPA) nano-formulation.

Methods

### Transmission Electron Microscopy

Transmission Electron Microscope (TEM) measurements were performed using a FEI Tecnai F30 300 kV FEG(s) TEM microscope. Carbon-coated copper grids were treated with oxygen plasma before deposition of the samples. The samples were deposited on the carbon grids for 1 min, and excess samples were wicked away. The samples were allowed to dry under ambient conditions. Samples were stained with uranyl acetate.

### Synthesis of Nanoparticles

Amphiphilic materials (3 mg each) were dissolved in dimethyl sulfoxide (DMSO) (1.5 mL) and was stirred at rt overnight. The solution (1.5 mL) was then added dropwise to nanopure water (1.5 mL) over a time period of 3 h. The solution was stirred for another 3 h and was then subjected to dialysis against nanopure water/PBS for 72 h to afford a NP solution. Following this, the particles were stored at 4° C. The particles were further analyzed using TEM, MALS and CD spectroscopy. The concentration of 2/6_7a in particles were measured by UV-Vis spectroscopy and HPLC analysis following overnight degradation using DMSO. Further, the kinetics and stability of the nanoparticles have been monitored using TEM over a period of eight weeks at 4° C.

### Example 3

### SPA Enhances Stability and Potency In Vitro

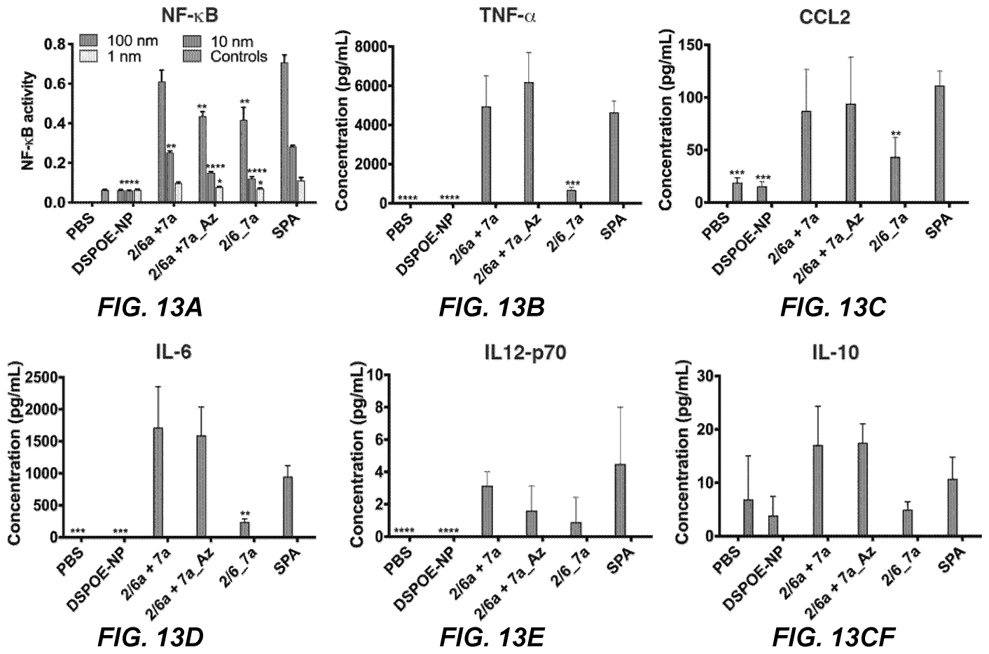

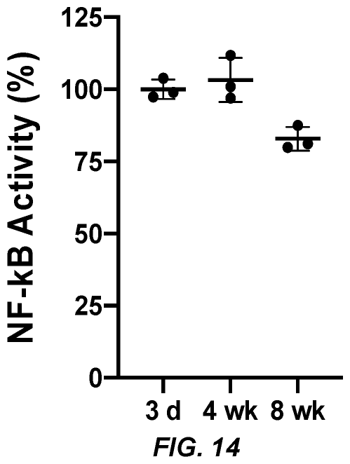

Long-term stability is an essential attribute of nano-medicines for clinical applications. As the SPA assembled into stable particles unchanged over two months, its immunological activity was evaluated via quantification of activity of NF-κB,³⁴ a cytosolic transcription factor that migrates to the nucleus on activation by pathogenic stimuli (e.g., TLR agonists). Studies on NF-κB activity were performed with RAW Blue macrophage reporter cells, derived from RAW 264.7 macrophages. A series of pro-inflammatory cytokines secreted on activation of Bone-marrow derived dendritic cells (BMDCs) by the agonists were analyzed. These assays indicated that the conjugation chemistry results in a modest decrease of NF-κB activity (FIG. 13A) and significant decrease in cytokine production by the heterodimers compared to the unlinked formulation. One explanation is the loss in conformational entropy upon binding due to presence of flexible linkers. SPA particles, by contrast, enhanced the activity of the conjugate (FIGS. 13B-F). This enhancement may be due to localized concentration-dependent activation or shape dependent stimulation of immune cells by the particles.³⁵ Further, no significant decrease in activity of the SPA particles was observed over eight weeks (FIG. 14). It was concluded that the SPA was both structurally and functionally stable when stored at 4° C.

Methods

### General

Ultrapure LPS was purchased from Invivogen (San Diego, Calif., USA). Cell culture media (DMEM, RPMI-1640), fetal bovine serum (FBS) and heat-inactivated fetal bovine serum (HI-FBS) were bought from Thermo Fisher Scientific. HEK Blue detection media was from Invivogen. Supplementary antibiotics were from Thermo Fisher Scientific and Invivogen. Cytokine bead array kit (Mouse Inflammation Kit) was from BD Biosciences. Quanti Blue reagent was purchased from Invivogen. RAW Blue and HEK Blue mTLR 4 cells were from Invivogen. RAW Blue and HEK Blue mTLR 4 cells were passaged when they reached 70% confluency.

### Cells

Raw Blue cells were cultured in DMEM media with 10% (v/v) FBS supplemented with Glucose (4.5 g/L), L-Glutamine (584 mg/L), Penicillin (50 μg/mL), Streptomycin (50 μg/mL), Normocin (100 μg/mL). 10% (v/v) heat-inactivated FBS was used in place of FBS for assays.

### Endotoxin Tests

Analysis of endotoxin contamination in the samples were conducted using HEK Blue TLR4 cells following manufacturer's protocol. Samples were confirmed endotoxin-free (<1 EU/mL). HEK Blue mTLR4 cells were cultured in DMEM media with 10% (v/v) FBS supplemented with Glucose (4.5 g/L), L-Glutamine (584 mg/L), Penicillin (50 μg/mL), Streptomycin (50 μg/mL), Normocin (100 μg/mL) along with HEK Blue selection antibiotic.

### RAW Blue Assay

NF-κB activity was monitored using RAW Blue cells. RAW-Blue cells (180 μL) were plated at a density of 0.55×10⁶ cells/mL in 96-well plates in DMEM media supplemented 10 with heat-inactivated FBS. Cells were incubated with agonists at various concentrations for 18 h following which 50 μL of the supernatant was transferred into a 96-well plate and SEAP activity was monitored with QUANTI-Blue reagent (InvivoGen) following manufacturers protocol by measuring absorbance at 620 nm. The SEAP activity serves as a measure of NF-κB activity by the cells.

### Bone Marrow-Derived Dendritic Cell Harvest & Culture

Bone marrow-derived dendritic cells (BMDCs) were harvested from 6-week-old C57BL/6 mice (Jackson Laboratory) following previous literature protocol.^{1,3} On day 6, BMDCs were released and plated on 96-well plates at a density of 1.1×10⁶ cells/mL (180 μL) and incubated with agonists in desired concentrations for 6 h following which the plates were centrifuged at 400×g and the supernatants were collected. The supernatants were diluted 2.5 times and the cytokine profile was analyzed using Mouse Inflammation kit (BD Biosciences) following manufacturers protocol. Raw data were analyzed and plotted using Prism 7 software. Each experiment was performed twice independently with three replicates for each experiment.

### Example 4

### SPA Formulation Reduces Toxicity and Enhances Efficacy In Vivo

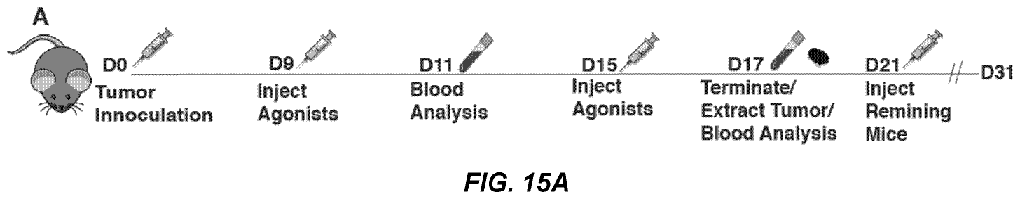

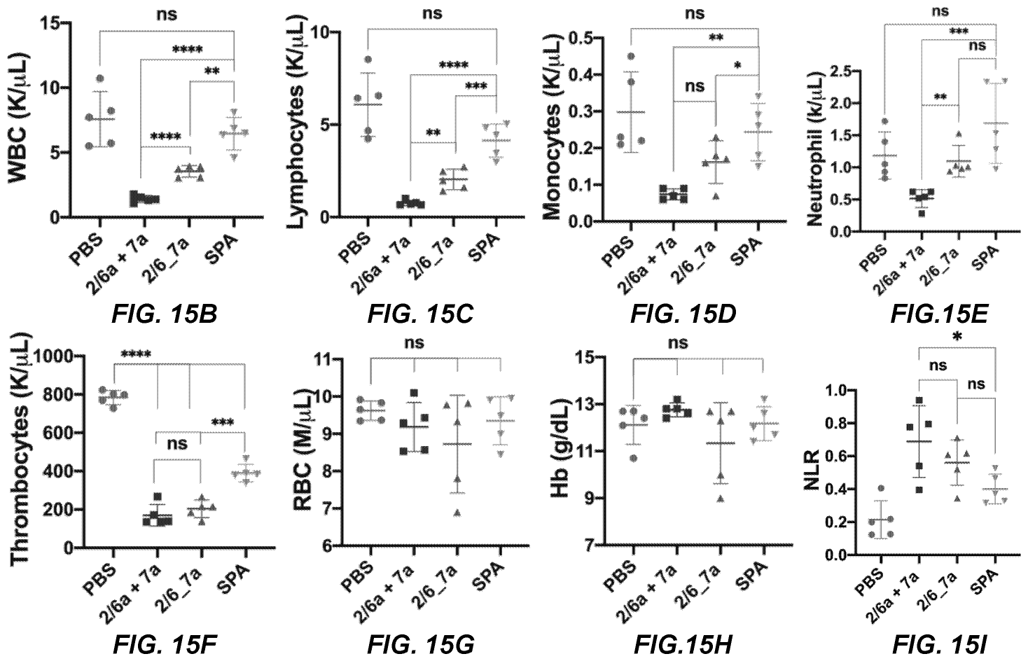

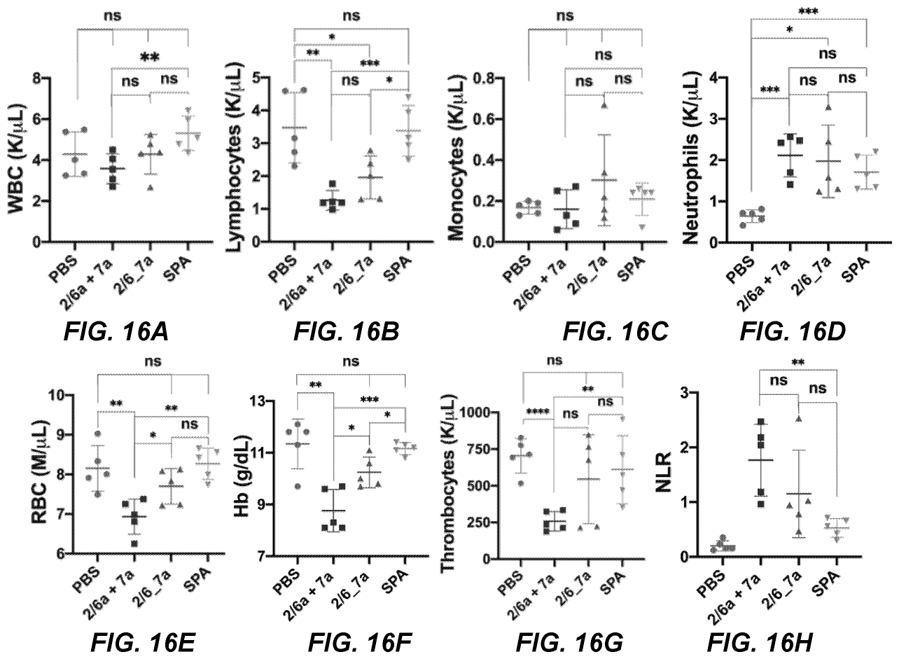

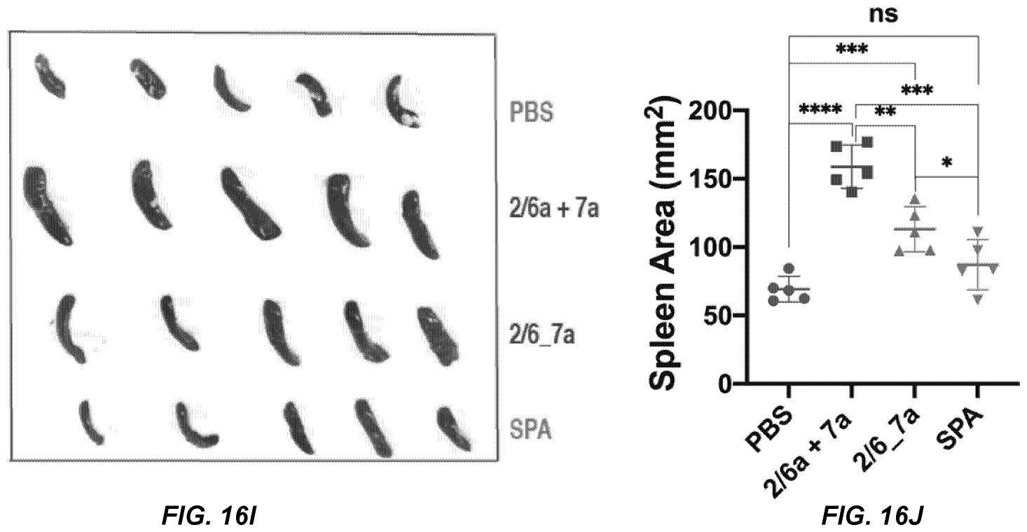

To evaluate the toxicity and efficacy of the formulated, linked TLR agonists, immunotherapy studies were conducted in a B16.F10 tumor challenge model. Many TLR agonist formulations have unacceptably high hematological toxicity at clinically relevant dosages.¹¹ Unlinked agonists have rapid systemic diffusion into the blood resulting in systemic cytokines.^{13,18} This diffusion leads to hematological toxicity resulting in decrease of WBC counts along with thrombocytopenia and hemolysis.^{13,36} It was hypothesized that both the linked agonist and SPA would be more capable in restricting immune activation at the site of injection and hence reduce toxicity. Formulations of PBS, unlinked TLR agonists, linked heterodimer and the SPA were tested. Each formulation comprised 17.5 nmole of each of the agonists injected peritumorally on day 9 post inoculation when the tumor volume reached about 100 mm³ and treatment was repeated on day 15 and day 21. Blood was collected two days post first and second injection and whole blood counts were analyzed. The results showed great differences between linked and unlinked and formulated TLR agonist (FIGS. 15A-I). Injection of unlinked agonists caused reduction of WBCs including neutrophils, lymphocytes, monocytes (FIGS. 15B-E). It also resulted in severe reduction of thrombocytes (FIG. 15F). Even though the linked agonists caused severe thrombocytopenia, the levels of WBCs including lymphocytes and neutrophils were statistically higher compared to unlinked formulation. The SPA had higher blood cell counts compared to both the linked and unlinked agonists. The SPA did not create any observed differences in terms of WBC counts compared to PBS samples and significantly reduced thrombocytopenia compared to linked or unlinked formulations in treated animals. Post second injection, the unlinked agonists demonstrated significant decrease in WBCs and lymphocytes compared to the SPA formulation (FIGS. 16A-D). All agonist-treated groups also demonstrated an enhancement in neutrophil population (FIG. 16D). It was further observed that the unlinked agonists consistently caused an enhanced neutrophil-to-lymphocyte ratio (NLR) (FIG. 151 and FIG. 16H) amongst agonist-treated groups. The NLR has been used as a biomarker for advanced melanoma and other solid tumors with high NLR being associated with poor prognosis.''' The unlinked agonists were also observed to induce significant thrombocytopenia (FIG. 16G) and hemolysis (FIG. 16E) accompanied with reduction of total hemoglobin (FIG. 16F) in the animals. This severe and sustained thrombocytopenia with unlinked formulation raises the risk of internal bleeding in treated groups. Further, hemolytic anemia may lead to various clinical symptoms.³⁹ Splenomegaly was also observed in animals administered with unlinked agonists indicating significant systemic immune activation (FIGS. 16I-J).⁴⁰ Mice in unlinked agonist group were also found to be highly lethargic post injection and displayed adverse clinical symptoms. The linked TLR agonists also resulted in enlarged spleen, though significantly lower than unlinked formulations. The SPA formulation, in contrast, did not induce splenomegaly indicating localized immune response due to improved PK/PD profile. These results indicated that the SPA caused significant reduction in systemic toxicity resulting from diffusion of admixed TLR agonists.

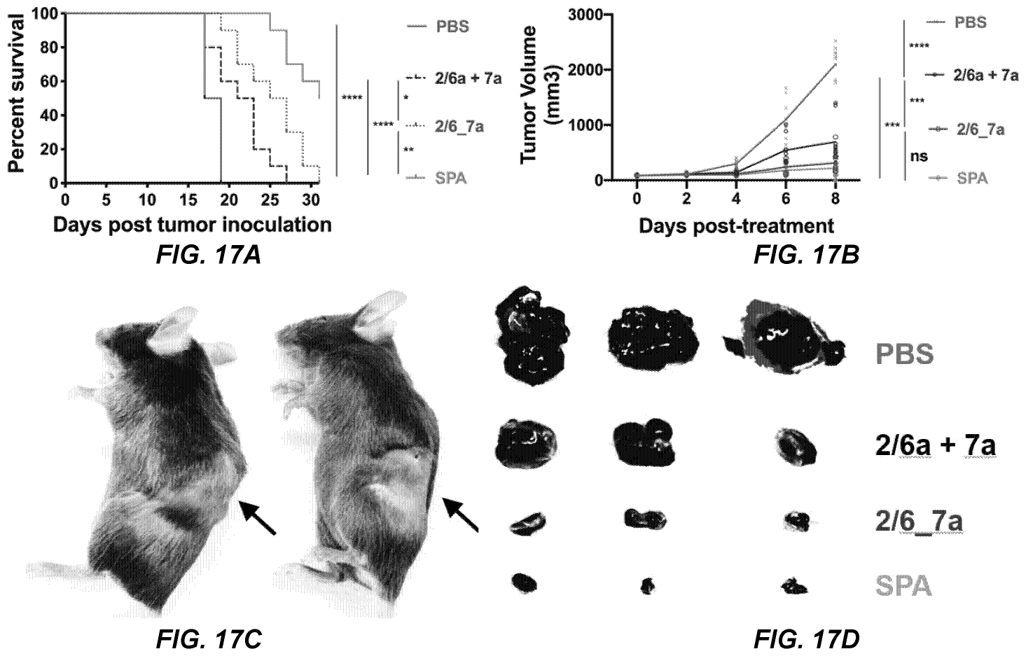

With the observation that the SPA formulation significantly reduced toxicity, its efficacy in the B16.F10 model was further evaluated. B16.F10 is a very poorly immunogenic and highly aggressive murine tumor model.⁴¹ Hence, the efficacy of the formulations in reducing tumor burden and prolonging survival over a period of 31 days was evaluated. All the formulations displayed significantly higher survival compared to PBS controls (FIG. 17A). Even though the linked formulation displayed lower activity in in vitro studies compared to unlinked agonists, it significantly enhanced efficacy in vivo. Further, the SPA formulation significantly reduced tumor burden and prolonged survival compared to other treatment groups (FIGS. 17A-D). While none of the animals completely rejected their tumors, the SPA formulation was observed to enhance survival beyond 31 days in 50% of challenged animals. The lower survival for the unlinked mice may be due to the severe toxicity induced by systemic diffusion of unlinked agonists resulting in severely comprised immune systems and acute thrombocytopenia in these animals.

To better understand the role of each of these formulations, tumors were isolated two days post second injection and analyzed for tumor-infiltrating immune cell populations. Both the linked agonist heterodimers and the SPA significantly enhanced the percentage of tumor-infiltrating leucocytes (TIL) in each tumor (FIG. 17E). Amongst various TILs, cytotoxic T-lymphocytes (CTLs) are known to reduce tumor volumes directly through the release of IFN-γ, granzymes, perforin and granulysin.⁴¹ The SPA significantly enhanced the percentage of tumor-infiltrating CD8+ cytotoxic T lymphocytes (CTLs) compared to other groups (FIG. 17F). Further, the SPA increased the percentage of infiltrating natural killer cells which plays a key role in activation of innate immunity and development of an adequate adaptive immune response (FIG. 17G).^{43,44} Such infiltration of CD8+ T cells and NK cells into the tumor microenvironment is associated with improved host survival and is critical for tumor-specific immunity.^{45,46} A decrease in surface expression of CD206, a canonical marker of M2-polarized macrophages, was observed, indicating significant repolarization for all the formulations compared to PBS controls (FIG. 17H). This result suggested the recruitment of macrophages with reduced immunosuppressive potential.⁴⁷ However, all the formulations also displayed an increase in the percentage of myeloid derived suppressive cells (MDSCs), considered an important indicator of immune-suppression, as these cells suppress tumoricidal activity of CTL and NK cells (FIG. 17I).⁴⁸ Treatment with SPA induced similar levels of MDSC activation compared to the unlinked formulation and reduced the percentage of MDSCs compared to the linked agonist formulation.

To further explore the functional activity of CD8 T-cells, splenocytes were stimulated ex vivo with B16.F10 melanoma cells. It was observed that SPA significantly enhanced the percentage of antigen specific IFN-γ and TNF-α secreting CD8 T-cells (FIGS. 17J-K). The percentage of dual IFN-γ and TNF-a secreting CD8+ splenocytes were also enhanced indicating enhanced anti-tumor functionality of SPA based therapy (FIG. 17L). Thus, it can be envisioned that the SPA provides a robust strategy to enhance anti-tumor efficacy while reducing toxicity.

Methods

### General

All animals (5-week-old) were purchased from Jackson Laboratory (JAX). Mice were housed in an AAALAC accredited animal facility. All animal procedures were performed under a protocol approved by the University of Chicago Institutional Animal Care and Use Committee (IACUC). All compounds were tested for endotoxin prior to use. The animals were allowed to rest for 7 days post receipt prior to injections.

### Immunotherapy Studies

0.5×10⁶ B16. F10 cells were injected subcutaneously into the flank of 6 week old mice (n=15 per group) in 100 μL of serum-free RPMI media. The tumor size was measured on alternating days. Tumor volumes were measured using the equation V=1/2×L×W×W. When the tumors reached a size of approx. 100 mm³ (day 9), treatment was started. Various formulations (17.5 nmoles of each agonists or PBS) were injected peritumorally every 6 days (day 15 and day 21). Mice were euthanized when the tumors reached 17.5 mm in any linear dimension. Five mice for each group were used for blood analysis. These mice were euthanized two days post second injection (day 17) and spleens and tumors were extracted for further analysis.

### Whole Blood Analysis

Two days post injection, blood was collected from animals by submandibular bleed in EDTA coated Eppendorf Tubes™ (Fisher Scientific). Samples were immediately analyzed for complete blood count (CBC) using a Hemavet 950 instrument (Drew Scientific).The instrument was fitted with a reagent pack obtained from the manufacturer. Prior to analysis a blank run and a quality control run (using manufacturer provided control sample) were performed to ensure optimal performance by the instrument. 20 μL of blood were injected for each analysis using a sample cycle of approximately two minutes.

### Tumor Extraction and Analysis

Two days post second injection, tumors were extracted from mice (n=5) of each group. Tumors were smashed and strained through a 70 μm cell strainer. 4×10⁶ cells from each tumor was used for analysis. Two sets of samples were made for each tumor. Cells were incubated with anti-CD16/32 in FACS buffer at 4° C. for 10 min., washed, and stained with fluorochrome labeled antibodies. The following dyes were used: FITC anti-CD45, PE anti-CD3, APC anti-CD8a, PE-Cy7 anti-CD49b (for CD8/NK analysis) and FITC anti-CD45, PE anti-CD1 lb, PerCP-Cy5.5 anti Gr-I, APC anti-F4/80, PE-Cy7 anti CD 206 (for MDSC and Macrophage polarization analysis). 4 x 10⁵ cells were analyzed for each sample using a Novocyte Flow Cytometer.

### Splenocyte Extraction and Analysis

Two days post second injection, spleens were extracted from mice (n=5) of each group. Tumors were smashed and strained through a 40 μm cell strainer and treated with ACK lysing buffer (Gibco). 4×10⁶ cells (400 μL) from each spleen were plated at a density of 10 M cells/mL. 5×10⁴B16.F10 cells were added to each culture. After stimulation for 1 h , Brefeldin were added to the cells and incubated for 10 hrs. following which cells were washed and incubated with anti-CD16/32 in FACS buffer at 4° C. for 10 mins. Cells were further processed and stained for cell surface markers and intracellular cytokines using Fixation/Permeabilization solution kit (BD Biosciences) following manufacturer's protocol. The following fluorochrome-conjugated antibodies were used: AF 488 anti-IFN-γ, PE anti-TNF-α, APC anti-CD8, APC/cy7 anti-CD3. 5×10⁴ cells were analyzed for each sample.

### Example 5

### Loading of Nanoparticles with Paclitaxel

### Synthesis of Nanoparticles

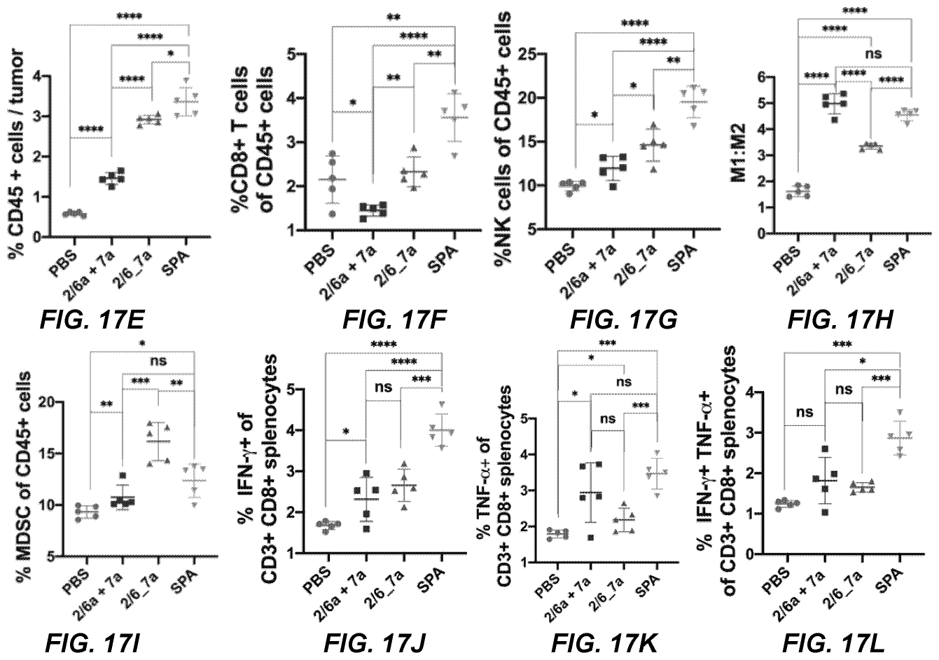

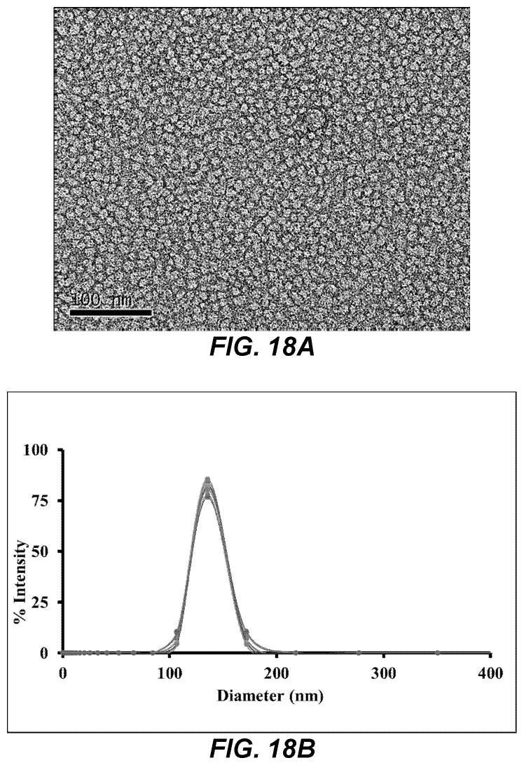

Amphiphilic block copolymer (OL-DSOPE) (6.0 mg) was dissolved in tetrahydrofuran (THF)/dimethyl formamide (DMF)/DMSO) (2.0 mL) and was stirred at room temperature overnight. Dissolved polymeric solution (2.0 mL) was then added dropwise to nanopure water (2.0 mL) over a time period of 4 hours. The solution was stirred for another 4 hours and then was subjected to dialysis against nanopure water for 72 h to afford a NP solution. Nanoparticle concentration: 1.0 mg/mL. The ratio (OL-DSOPE) (1:10) was self-assembled and resulted in particles (10±1.5 nm) as shown in FIGS. 18A and 18B. FIG. 18A shows transmission electron microscopy images of nanoparticles derived from OL-DSPOE (1:10) (10±1.5 nm). FIG. 18B shows the hydrodynamic diameter Dh of the nanoparticles (150±5.6 nm).

### Drug Loading

To the nanoparticle solution (1.0 mg/mL) in a 15 mL vial was added Paclitaxel (PTXL) (3.0 mg/mL in CH₂Cl₂). The solution was stirred for 18 h with the vial being open. Insoluble PTXL was removed by centrifugation (6000 rpm×10 min) in a centrifugal filter device (Amicon® Ultr-4, mwco=3.0 kDa, Merck KGaA, Germany). The PTXL-loaded NPs were then reconstituted to a final volume of 4 mL. After adding 3 folds of methanol (v/v), the UV-vis absorbance at 239 nm was measured and the amount of incorporated PTXL was determined using a calibration curve of PTXL of varying concentration in MeOH/PBS 3:1. The Drug Loading Efficiency (DLE) was calculated according to the following equation: Drug loading efficiency

$$( DLE ) = ( \frac{Df}{Dt} \times 100 ) \%$$

where [Drug]f is the concentration of paclitaxel (PTXL) in the nanoparticles and [Drug]t is the theoretical concentration of drug (i.e, the total amount of paclitaxel added initially). The drug loading efficiency of this process under the described conditions was determined to be 3%.

### Example 6

### Systemic Toxicity Analysis

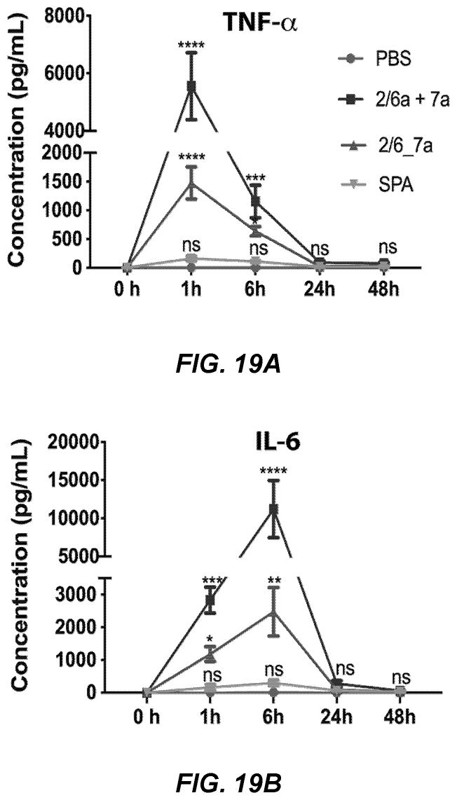

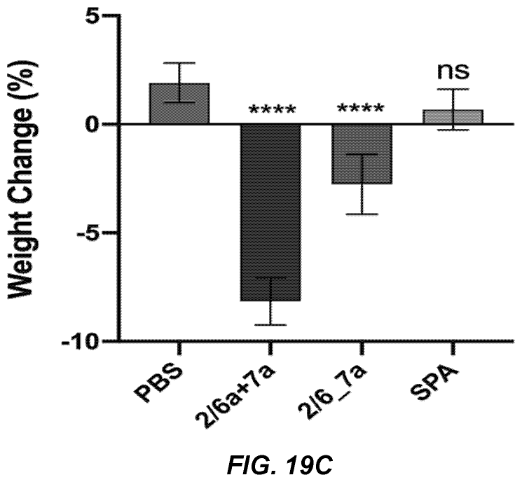

To understand the effect of SPA formulation in reducing toxicity, studies were performed to analyze for secretion of systemic cytokines following injection of TLR agonists. Systemic cytokines can be secreted in the blood as a result of systemic diffusion of immune agonists from the site of injection, thereby creating off-target toxicity.^{49,50} In this study, tumor-bearing mice were treated with the various agonist formulations as described in Example 5. Blood was collected by submandibular bleed at 2, 6, 24, and 48 h post injection of agonists at the tumor site on day 9 and analyzed for level of serum cytokines TNF-α and IL-6. SPA formulation did not induce significant systemic cytokine secretion, indicating better localization (FIGS. 19A and 19B). In contrast, treatment with unlinked agonist formulation (2/6+7a) or linked agonist formulation (2/6_7a) significantly enhanced the generation of systemic cytokines in blood indicating diffusion of agonists from the site of injection (FIGS. 19A and 19B). The generation of significant amounts of systemic cytokines in animals treated with unlinked agonists or the linked heterodimer also resulted in significant weight loss (FIG. 19C). In contrast, no weight loss was observed in mice treated with the SPA formulation (FIG. 19C). The results demonstrated reduced toxicities with the SPA formulation.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The references recited in the application, to the extent that they provide exemplary procedural or other details supplementary to those set forth herein, are specifically incorporated herein by reference.

### REFERENCES

The following references and the publications referred to throughout the specification, to the extent that they provide exemplary procedural or other details supplementary to those set forth herein, are specifically incorporated herein by reference.
1. Pardoll, D. M. Immunology beats cancer: a blueprint for successful translation. Nat. Immunol. 13, 1129-1132 (2012).
2. Ribas, A. & Wolchok, J. D. Cancer immunotherapy using checkpoint blockade. Science 359, 1350-1355 (2018).
3. Sharma, P. & Allison, J. P. The future of immune checkpoint therapy. Science 348, 56-61 (2015).
4. Sharma, P., Hu-Lieskovan, S., Wargo, J. A. & Ribas, A. Primary, Adaptive, and Acquired Resistance to Cancer Immunotherapy. Cell 168, 707-723 (2017).
5. Vanpouille-Box, C. et al. Trial watch: Immune checkpoint blockers for cancer therapy. Oncoimmunology 6, e1373237 (2017).
6. Jenkins, R. W., Barbie, D. A. & Flaherty, K. T. Mechanisms of resistance to immune checkpoint inhibitors. Br. J. Cancer 118, 9-16 (2018).
7 Palm, N. W. & Medzhitov, R. Pattern recognition receptors and control of adaptive immunity. Immunol. Rev. 227, 221-233 (2009).
8. Thompson, M. R., Kaminski, J. J., Kurt-Jones, E. A. & Fitzgerald, K. A. Pattern recognition receptors and the innate immune response to viral infection. Viruses 3, 920-40 (2011).
9. Kawasaki, T. & Kawai, T. Toll-like receptor signaling pathways. Front. Immunol. 5, 461 (2014).
10. Tom, J. K. et al. Applications of Immunomodulatory Immune Synergies to Adjuvant Discovery and Vaccine Development. Trends Biotechnol. 37, 373-388 (2019).
11. Engel, A. L., Holt, G. E. & Lu, H. The pharmacokinetics of Toll-like receptor agonists and the impact on the immune system. Expert Review of Clinical Pharmacology 4, 275-289 (2011).
12. Hanson, M. C. et al. Nanoparticulate STING agonists are potent lymph node-targeted vaccine adjuvants. J. Clin. Invest. 125, 2532-2546 (2015).
13. Nuhn, L. et al. Nanoparticle-Conjugate TLR7/8 Agonist Localized Immunotherapy Provokes Safe Antitumoral Responses. Adv. Mater. 30, 1803397 (2018).
14. Albin, T. J. et al. Linked Toll-Like Receptor Triagonists Stimulate Distinct, Combination-Dependent Innate Immune Responses. ACS Cent. Sci. acscentsci.8b00823 (2019). doi:10.1021/acscentsci.8b00823
15. Zhao, B., Vasilakos, J. P., Tross, D., Smirnov, D. & Klinman, D. M. Combination therapy targeting toll like receptors 7, 8 and 9 eliminates large established tumors. J. Immunother. Cancer 2, 12 (2014).
16. Ignacio, B. J., Albin, T. J., Esser-Kahn, A. P. & Verdoes, M. Toll-like Receptor Agonist Conjugation: A Chemical Perspective. Bioconjug. Chem. 29, 587-603 (2018).
17. Macedo, A. B. et al. Dual TLR2 and TLR7 agonists as HIV latency-reversing agents. JCI Insight 3, (2018).
18. Lynn, G. M. et al. In vivo characterization of the physicochemical properties of polymer-linked TLR agonists that enhance vaccine immunogenicity. Nat. Biotechnol. 33, 1201-1210 (2015).
19. Black, M. et al. Self-Assembled Peptide Amphiphile Micelles Containing a Cytotoxic T-Cell Epitope Promote a Protective Immune Response In Vivo. Adv. Mater. 24, 3845-3849 (2012).
20. Lueckheide, M., Vieregg, J. R., Bologna, A. J., Leon, L. & Tirrell, M. V. Structure— Property Relationships of Oligonucleotide Polyelectrolyte Complex Micelles. Nano Lett. 18, 7111-7117 (2018).
21. Rudra, J. S., Tian, Y. F., Jung, J. P. & Collier, J. H. A self-assembling peptide acting as an immune adjuvant. Proc. Natl. Acad. Sci. U. S. A. 107, 622-7 (2010).
22. Mora-Solano, C. et al. Active immunotherapy for TNF-mediated inflammation using self-assembled peptide nanofibers. Biomaterials 149, 1-11 (2017).
23. Larché, M. & Wraith, D. C. Peptide-based therapeutic vaccines for allergic and autoimmune diseases. Nat. Med. 11, S69-S76 (2005).
24. Gröschel, A. H. & Müller, A. H. E. Self-assembly concepts for multicompartment nanostructures. Nanoscale 7, 11841-11876 (2015).
25. Li, Y., Wang, Y., Huang, G. & Gao, J. Cooperativity Principles in Self-Assembled Nanomedicine. Chem. Rev. 118, 5359-5391 (2018).
26. Sato, K., Hendricks, M. P., Palmer, L. C. & Stupp, S. I. Peptide supramolecular materials for therapeutics. Chem. Soc. Rev. 47, 7539-7551 (2018).
27. Cui, H., Chen, Z., Zhong, S., Wooley, K. L. & Pochan, D. J. Block Copolymer Assembly via Kinetic Control. Science (80-.). 317, 647-650 (2007).
28. Cai, C., Wang, L. & Lin, J. Self-assembly of polypeptide-based copolymers into diverse aggregates. Chem. Commun. 47, 11189 (2011).
29. Castelletto, V., Newby, G. E., Zhu, Z., Hamley, I. W. & Noirez, L. Self-Assembly of PEGylated Peptide Conjugates Containing a Modified Amyloid β-Peptide Fragment. Langmuir 26, 9986-9996 (2010).
30. Li, Z., Kesselman, E., Talmon, Y., Hillmyer, M. A. & Lodge, T. P. Multicompartment Micelles from ABC Miktoarm Stars in Water. Science (80-.). 306, 98 LP-101 (2004).
31. Maiti, S., Manna, S., Shen, J., Esser-Kahn, A. P. & Du, W. Mitigation of Hydrophobicity-Induced Immunotoxicity by Sugar Poly(orthoesters). J. Am. Chem. Soc. 141, 4510-4514 (2019).
32. Reddy, S. T., Rehor, A., Schmoekel, H. G., Hubbell, J. A. & Swartz, M. A. In vivo targeting of dendritic cells in lymph nodes with poly(propylene sulfide) nanoparticles. J. Control. Release 112, 26-34 (2006).
33. Fifis, T. et al. Size-Dependent Immunogenicity: Therapeutic and Protective Properties of Nano-Vaccines against Tumors. J. Immunol. 173, 3148-3154 (2004).
34. Gilmore, T. D. Introduction to NF-κB: players, pathways, perspectives. Oncogene 25, 6680-6684 (2006).
35. Kumar, S., Anselmo, A. C., Banerjee, A., Zakrewsky, M. & Mitragotri, S. Shape and size-dependent immune response to antigen-carrying nanoparticles. J. Control. Release 220, 141-148 (2015).
36. Cauwels, A. et al. Delivering Type I Interferon to Dendritic Cells Empowers Tumor Eradication and Immune Combination Treatments. Cancer Res. 78, 463-474 (2018).
37. Ma, J. et al. Neutrophil-to-lymphocyte Ratio (NLR) as a predictor for recurrence in patients with stage III melanoma. Sci. Rep. 8, 4044 (2018).
38. Gandini, S. et al. Prognostic significance of hematological profiles in melanoma patients. doi:10.1002/ijc.30215
39. Go, R. S., Winters, J. L. & Kay, N. E. How I treat autoimmune hemolytic anemia. Blood 129, 2971-2979 (2017).
40. Chapman, J. & Azevedo, A. M. Splenomegaly. StatPearls (StatPearls Publishing, 2019).
41. Wang, J., Saffold, S., Cao, X., Krauss, J. & Chen, W. Eliciting T cell immunity against poorly immunogenic tumors by immunization with dendritic cell-tumor fusion vaccines. J. Immunol. 161, 5516-24 (1998).
42. Durgeau, A., Virk, Y., Corgnac, S. & Mami-Chouaib, F. Recent Advances in Targeting CD8 T-Cell Immunity for More Effective Cancer Immunotherapy. Front. Immunol. 9, 14 (2018).
43. Souza-Fonseca-Guimaraes, F., Cursons, J. & Huntington, N. D. The Emergence of Natural Killer Cells as a Major Target in Cancer Immunotherapy. Trends Immunol. 40, 142-158 (2019).
44. Tarazona, R., Duran, E. & Solana, R. Natural Killer Cell Recognition of Melanoma: New Clues for a More Effective Immunotherapy. Front. Immunol. 6, 649 (2015).
45. Melero, I., Rouzaut, A., Motz, G. T. & Coukos, G. T-Cell and NK-Cell Infiltration into Solid Tumors: A Key Limiting Factor for Efficacious Cancer Immunotherapy. Cancer Discov. 4, 522-526 (2014).
46. Malhotra, A. & Shanker, A. NK cells: immune cross-talk and therapeutic implications. Immunotherapy 3, 1143-66 (2011).
47. Rodell, C. B. et al. TLR7/8-agonist-loaded nanoparticles promote the polarization of tumour-associated macrophages to enhance cancer immunotherapy. Nat. Biomed. Eng. 2, 578-588 (2018).
48. Gabrilovich, D. I. & Nagaraj, S. Myeloid-derived suppressor cells as regulators of the immune system. Nat. Rev. Immunol. 9, 162-74 (2009).
49. Moser, B.; Steinhardt, R.; Escalante-Buendia, Y.; Boltz, D.; Barker, K.; Cassaidy, B.; Rosenberger, M.; Yoo, S.; McGonnigal, B.; Esser-Kahn, A. Increased vaccine tolerability and protection via NFκB modulation. Science Advances 2020, 6 (37), eaaz8700.
50. Wu, T. Y.-H.; Singh, M.; Miller, A. T.; De Gregorio, E.; Doro, F.; D′Oro, U.; Skibinski, D. A.; Mbow, M. L.; Bufali, S.; Herman, A. E. Rational design of small molecules as vaccine adjuvants. Science Translational Medicine 2014, 6 (263), 263ra160.

1. A nanoparticle comprising:
(a) an amphiphile of formula (I):
A-B-C (I)
wherein:
A is an amphiphilic group;
B is a hydrophilic linker; and
C is a first hydrophobic group; and
(b) a nanoparticle co-assembly agent of formula (II):
X-(Y-Z)ₘ (II)
wherein:
X is a functionalized hydrophilic polymer;
Y is a co-assembly agent linker;
Z is a second hydrophobic group; and
m is an integer ranging from 1 to 500.

2. The nanoparticle of claim 1, wherein the nanoparticle is a self-assembled nanoparticle.

3. The nanoparticle of claim 1 or 2, wherein the amphiphile and the nanoparticle co-assembly agent are not covalently attached to each other.

4. The nanoparticle of any of claims 1-3, wherein A comprises a polypeptide.

5. The nanoparticle of any of claims 1-4, wherein the polypeptide is a hydrophilic polypeptide.

6. The nanoparticle of any of claims 1-5, wherein A comprises an amphiphilic pattern recognition receptor (PRR) agonist.

7. The nanoparticle of claim 6, wherein the amphiphilic PRR agonist is a NOD-like receptor agonist, a RIG-I-like receptor agonist, a STING agonist, or a TLR agonist.

8. The nanoparticle of claim 7, wherein the amphiphilic PRR agonist is an amphiphilic toll-like receptor (TLR) agonist.

9. The nanoparticle of claim 8, wherein the amphiphilic TLR agonist is a TLR2/6 agonist.

10. The nanoparticle of claim 8 or 9, wherein the amphiphilic TLR agonist is Pam₂CSK₄, Pam₃CSK₄, MALP-2, or FSL-1,

11. The nanoparticle of claim 10, wherein the amphiphilic TLR agonist is Pam₂CSK₄.

12. The nanoparticle of any of claims 1-11, wherein C comprises a hydrophobic PRR agonist.

13. The nanoparticle of claim 12, wherein the hydrophobic PRR agonist is a NOD-like receptor agonist, a RIG-I-like receptor agonist, a STING agonist, or a TLR agonist.

14. The nanoparticle of claim 13, wherein the hydrophobic PRR agonist is a hydrophobic TLR agonist.

15. The nanoparticle of claim 14, wherein the hydrophobic TLR agonist is a TLR7 agonist or a TLR8 agonist.

16. The nanoparticle of claim 14 or 15, wherein the hydrophobic TLR agonist is 2Bxy.

17. The nanoparticle of any of claims 1-16, wherein B comprises at least one of a maleimide moiety, a polyethylene glycol (PEG) moiety, and a triazole moiety.

18. The nanoparticle of claim 17, wherein B comprises a PEG moiety, and wherein the PEG moiety has between 3 and 7 ethyleneoxy units.

19. The nanoparticle of claim 18, wherein the PEG moiety has 5 ethyleneoxy units.

20. The nanoparticle of any of claims 1-17, wherein the amphiphile is of formula (III): 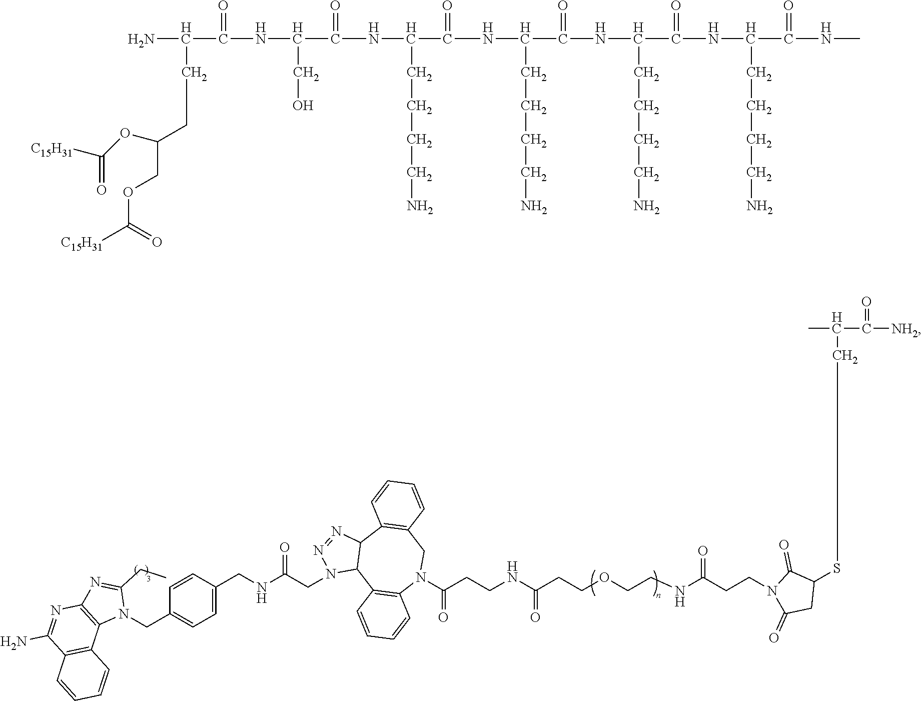
wherein n is an integer ranging from 3 to 7.

21. The nanoparticle of any of claims 1-20, wherein the amphiphile is of formula (IV):

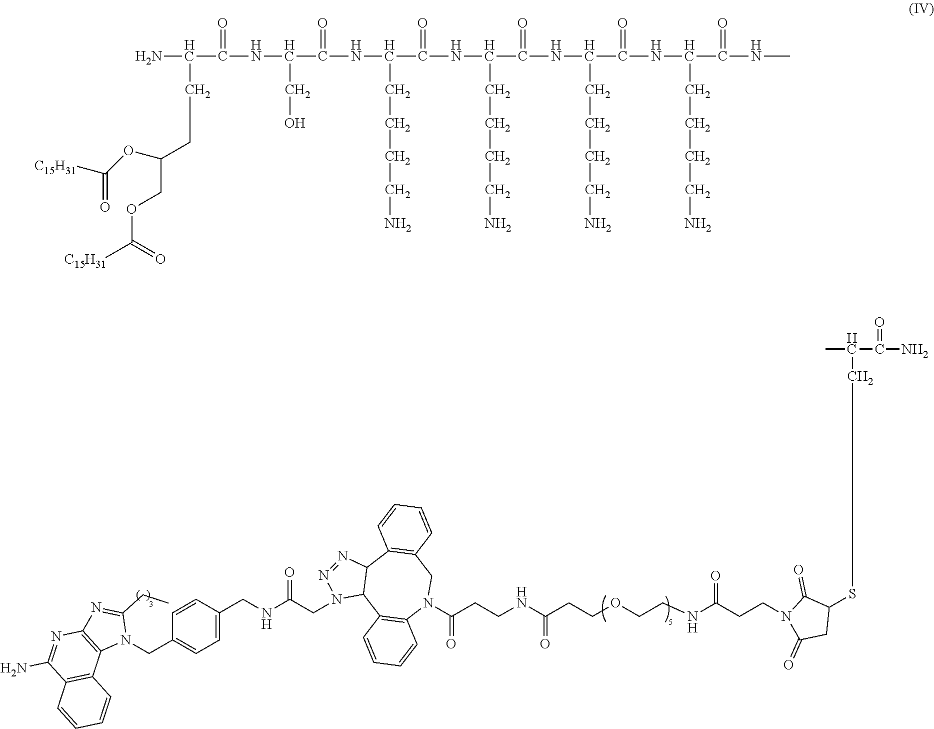

22. The nanoparticle of any of claims 1-21, wherein Xis a functionalized PEG or a functionalized polysaccharide.

23. The nanoparticle of any of claims 1-22, wherein Xis a pegylated polysaccharide or a monosaccharide poly(orthoester).

24. The nanoparticle of any of claims 1-23, wherein Y comprises at least one of a maleimide moiety, a PEG moiety, and a triazole moiety.

25. The nanoparticle of claim 24, wherein Y comprises a triazole moiety.

26. The nanoparticle of any of claims 1-25, wherein Z is an alkyl or olefinic group having at least 10 carbon atoms.

27. The nanoparticle of claim 26, wherein Z is an oleyl group.

28. The nanoparticle of any of claims 1-27, wherein the nanoparticle co-assembly agent is of formula (V): 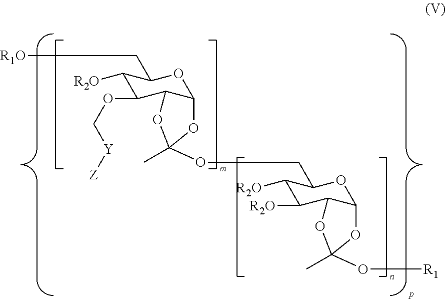
wherein:
R₁ is alkyl, acyl, or H;
R₂ is alkyl, acyl, or H;
m is an integer ranging from 1 to 10;
n is an integer ranging from 0 to 10;
Y is the co-assembly agent linker;
Z is the second hydrophobic group; and
p is an integer ranging from 2 to 500.

29. The nanoparticle of claim 28, wherein the ratio of m:n ranges from 1:0 to 1:10.

30. The nanoparticle of claim 29, wherein the ratio of m:n is 1:5.

31. The nanoparticle of any of claims 1-30, wherein the nanoparticle co-assembly agent is of formula (VI):

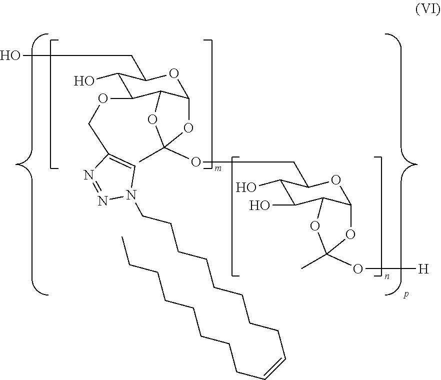

32. The nanoparticle of any of claims 1-31, wherein the nanoparticle is capable of inducing an immune response in an individual.

33. A method of making the nanoparticle of any of claims 1-32 comprising providing the amphiphile and the nanoparticle co-assembly agent in a salt solution.

34. The method of claim 33, wherein the salt solution is a balanced salt solution.

35. The method of claim 33 or 34, wherein the salt solution is a buffered salt solution.

36. The method of any of claims 33-35, wherein the salt solution is phosphate buffered saline.

37. The method of any of claims 33-36, wherein the method comprises providing the amphiphile and the nanoparticle co-assembly agent in the salt solution for at least 24 hours.

38. The method of any of claims 33-37, wherein the method further comprises subjecting the salt solution to dialysis.

39. A method of delivering an agent to an individual, the method comprising providing the nanoparticle of any of claims 1-32 to the individual, wherein the amphiphile comprises the agent.

40. The method of claim 39, wherein the nanoparticle is provided intravenously.

41. The method of claim 39 or 40, wherein the agent is an imaging agent.

42. The method claim 41, wherein the imaging agent is a fluorescent agent, a chemiluminescent agent, or a radiocontrast agent.

43. The method of claim 39 or 40, wherein the agent is a therapeutic agent.

44. The method of claim 43, wherein the therapeutic agent is an anti-viral agent, a chemotherapeutic, an immunotherapeutic, or an immunostimulatory agent.

45. The method of any of claims 39-43, wherein the agent is a PRR agonist.

46. The method of any of claims 39-45, wherein the agent is a TLR agonist.

47. The method of any of claims 39-46, further comprising providing an additional agent, wherein the amphiphile comprises the additional agent.

48. The method of claim 47, wherein the additional agent is a PRR agonist.

49. The method of claim 47 or 48, wherein the additional agent is a TLR agonist.

50. The method of any of claims 39-49, wherein the method comprises treating a condition in the individual.

51. The method of claim 50, wherein the condition is a viral infection, a neoplasm, an allergic disorder, or an autoimmune condition.

52. The method of claim 50 or 51, wherein the method comprises providing an effective amount of the nanoparticle to the individual to treat the condition.

53. The method of any of claims 50-52, wherein the method further comprises providing a therapy.

54. The method of any of claims 50-53, wherein the nanoparticle and the therapy are provided to the individual substantially simultaneously.

55. The method of any of claims 50-54, wherein the nanoparticle and the therapy are provided to the individual sequentially.

56. The method of any of claims 50-55, wherein the therapy is an anti-viral therapy, a chemotherapy, an immunotherapy, a radiotherapy, or a vaccine.

57. A polymer of formula (V): 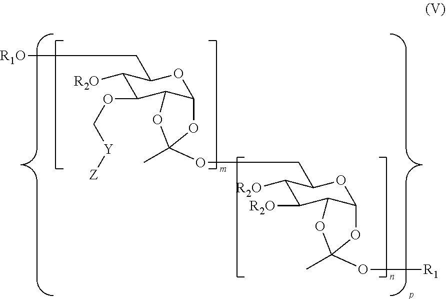
wherein:
R₁ is alkyl, acyl, or H;
R₂ is alkyl, acyl, or H;
m is an integer ranging from 1 to 10;
n is an integer ranging from 0 to 10;
Y is a linker;
Z is a hydrophobic group; and
p is an integer ranging from 2 to 500.

58. The polymer of claim 57, wherein Y includes at least one of a maleimide moiety, a PEG moiety, and a triazole moiety.

59. The polymer of claim 58, wherein Y comprises a triazole moiety.

60. The polymer of any of claims 57-59, wherein Z is an alkyl or acyl group having at least 10 carbon atoms.

61. The polymer of claim 60, wherein the alkyl or acyl group further includes at least one degree of unsaturation.

62. The polymer of claim 60, wherein Z is an oleyl group.

63. The polymer of any of claims 57-62, wherein the ratio of m:n ranges from 1:0 to 1:10.

64. The polymer of claim 63, wherein the ratio of m:n is 1:5.

65. The polymer of any of claims 57-62, wherein n is zero.

66. The polymer of claim 58, wherein the PEG moiety comprises from 2 to 20 ethyleneoxy units.

67. The polymer of any of claims 57-63, wherein the polymer is of formula (VI):

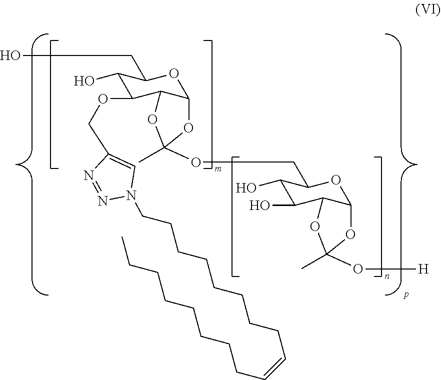

68. A pharmaceutical composition comprising (a) the nanoparticle of any of claims 1-32 or the polymer of any of claims 57-67 and (b) a pharmaceutically acceptable excipient.
